(19) United States
(12) Patent Application Publication
(10) Pub. No.: US 2024/0342223 A1
(43) Pub. Date: Oct. 17, 2024

(54) THERAPEUTIC AND PREVENTIVE COMPOSITIONS

(71) Applicant: Cellnutrition Ltd, Oranmore, Co. Galway (IE)

(72) Inventors: John Kelleher, Oranmore, Co. Galway (IE); Rania El Helou, Oranmore, Co. Galway (IE)

(21) Appl. No.: 18/701,070

(22) PCT Filed: Oct. 14, 2022
(86) PCT No.: PCT/EP2022/078687

§ 371 Date: Apr. 12, 2024

(30) Foreign Application Priority Data
Oct. 15, 2021 (GB) 2114802.8
Mar. 29, 2022 (GB) 2204461.4

(51) Int. Cl. A61K 35/644; A61K 35/08; A61P 31/14; A61P 31/20; A61P 31/22

(52) U.S. Cl. CPC A61K 35/644; A61K 35/08; A61P 31/14; A61P 31/20; A61P 31/22

(57) ABSTRACT

Provided are compositions comprising a bee-derived component and a marine plasma, and methods of preparation thereof, in particular wherein the bee-derived component comprises or is derived from zabrus. These compositions may be used in the treatment or prevention of disease, especially bacterial, viral, fungal, and/or parasitic diseases.

### TECHNICAL FIELD

The invention is in the field of therapeutic compositions and methods of producing the same.

### BACKGROUND

Despite lacking the antibody-based adaptive immune system found in mammals, bees and other insects have a well-developed innate immune system. Indeed, more recent evidence imply that the ‘innate’ immune system of insects is more robust and specific than the name is generally used to indicate. The concept of ‘immune priming’ in insects indicates that immune protection can be triggered in response to dead, harmless or sublethal microbe challenge, and can maintain and persist over time, and even across generations in bees (Cooper and Eleftherianos, “Memory and Specificity in the Insect Immune System: Current Perspectives and Future Challenges” Front. Immunol., 9 May 2017). This has obvious parallels with the specificity and memory of the supposedly unique mammalian adaptive immune system, and with the concept of vaccine inoculation.

It is thought that honeybee activity results in the take-up of environmental contaminants, including genetic material from pathogenic and other sources. Transmission of these pathogenic molecules has been found to be biologically active and triggers gene knockdown and immunity that lasts into adulthood. Transposable elements, non-coding RNA, as well as bacteria, fungi, and viruses results in an inherent property of honeybees to share immune-relevant among individuals and generations. Findings suggest that this may play a role in social immunity and signalling between members of the hive.

Antiviral defence mechanisms in the bee are varied in the pathways, cellular and extracellular effectors used. Such mechanisms include RNA interference (RNAi), endocytosis, melanization, encapsulation, autophagy, and conserved immune pathways including Jak/STAT (Janus kinase/signal transducer and activator of transcription), JNK (c-Jun N-terminal kinase), MAPK (mitogen-activated protein kinases) and the NF-κB mediated Toll and Imd (immune deficiency) pathways (McMenamin et al, “Honey Bee and Bumble Bee Antiviral Defense”, Viruses 2018).

Seawater, and/or solutions formed from its dilution, sometimes called ocean plasma or marine plasma, have been therapeutically used for over 125 years (Passebecq and Soulier, “Comparative Study of the Therapeutic Properties of Seawater Preparations” https://oceanplasma.org/documents/passbecsoulier-e.html). Evidence for the efficacy of saltwater in various therapeutic applications include recent studies indicating that saline irrigation and gargling are effective against the common cold (Ramalingam et al. “A pilot, open labelled, randomised controlled trial of hypertonic saline nasal irrigation and gargling for the common cold” Scientific Reports 2019).

Prophylactic approaches to prevent pathogenic invasion or improve the body's response to such challenge, such as vaccination, are a preferred means of reducing the impact of infectious disease, and are particularly effective against many bacterial and viral pathogens. However, in some instances, vaccines cannot provide the necessary immunity for prevention, for example in the case of Human Immunodeficiency Virus (HIV), eukaryotes such as the malarial parasite, and more generally in populations with reduced immune function, such as the elderly.

There is a need to provide methods, compositions, and methods for producing compositions for antimicrobial use. In particular, it is desired to provide means to prevent, ease, or relieve the symptoms of viral, bacterial, fungal, or parasitic infection in humans or animals. Preferably, these methods should involve minimal side effects, should be adaptable to specific pathogens, and should reduce the risk of the development of resistance.

### SUMMARY OF THE INVENTION

In one aspect, the invention provides a composition comprising a bee-derived component and a marine plasma.

The bee-derived component may comprise a product of apiculture, such as one or more of zabrus, honeycomb, bee venom, honey, royal jelly, propolis and pollen, suitably zabrus. The bee-derived component may be derived from a product of apiculture, such as one or more of zabrus, honeycomb, honey, royal jelly, propolis and pollen, suitably zabrus. The bee-derived component may comprise lysozyme, typically lysozyme derived from zabrus. The bee-derived component may have a reduced content of hydrophobic components compared to the input zabrus, honeycomb, bee venom, honey, royal jelly, propolis and/or pollen. The bee-derived component may have a reduced content of sugars compared to the input zabrus, honeycomb, bee venom, honey, royal jelly, propolis and/or pollen.

The composition may be formulated as a nasal spray, injectable serum, dermal patch, eyedrops and/or for oral administration.

The marine plasma may comprise seawater diluted with water, which may be at a ratio of 25% to 30% seawater, and 70% to 75% water, suitably approximately 29% seawater and 71% water.

In one aspect, the invention provides a method of making a composition, the method comprising providing a bee-derived component and combining the bee-derived component with a fluid comprising marine plasma.

The method may further comprise diluting seawater with water to produce the fluid comprising marine plasma. The method may further comprise obtaining seawater from below one or more plankton blooms in the sea, typically between about 25 and about 35 metres; and suitably around 30 metres below such plankton blooms. The seawater may be filtered, suitably microfiltered, typically double-cold micro-filtered. Filtration may take place through a micro-porous porcelain filter rated at 0.22 microns. The seawater may be diluted with purified, distilled, or reverse osmosis water, or with weakly mineralised spring water. The ratio of seawater to water in the marine plasma may be approximately 29% seawater and 71% water.

The bee-derived component may comprise one or more of zabrus, honeycomb, bee venom, honey, royal jelly, propolis and pollen. The bee-derived material may be derived from one or more of zabrus, honeycomb, bee venom, honey, royal jelly, propolis and pollen as raw material. The method may comprise processing one or more raw materials to remove at least a portion of the hydrophobic components thereof. Such removal may comprise disintegrating the one or more raw materials in water and centrifuging the resultant suspension. The method may comprise processing one or more raw materials to remove at least a portion of the sugar content thereof. Such removal may comprise using a polar solvent to precipitate sugars from the processed zabrus, honeycomb, bee venom, honey, royal jelly, propolis and/or pollen, typically wherein the polar solvent comprises acetone.

The method may further comprise rearing bees as a source of bee-derived components. The bees may be provided with a nectar feed comprising farnesol. The bees may be provided with a feed comprising linden nectar. The bees may be reared in a beehive, which may be treated with a fortification method. The fortification method may involve the application of rosin, or a coating comprising rosin on the outside of the hive. In some embodiments, one or more applications of 30% turpentine and 70% rosin, is used for hive fortification. The coating may be applied as soon as a hive is placed in a suitable location.

The method may further comprise obtaining one or more of zabrus, bee venom, honey, royal jelly, propolis and pollen, suitably zabrus, from the bees which have been reared or from their environment, such as a beehive.

In a further aspect, the invention provides a composition comprising a product of apiculture, such as a bee-derived component derived from zabrus, and methods for producing such compositions. Such compositions may be further defined or produced as described for the other aspects described above. Such compositions may further comprise water, saline solution and/or marine plasma.

In a yet further aspect, the invention provides methods of treating or preventing disease which include treating a subject in need thereof with one or more compositions as described above, or one or more compositions produced by methods as described above.

In a still further aspect, the compositions as described above, or produced by methods as described above, may be for use in medicine, typically for use in methods of treating or preventing disease.

In a further aspect, the invention provides uses of compositions as described above, or produced by methods as described above, in the manufacture of a medicament

In the above aspects, the diseases to be treated or prevented include bacterial, viral, fungal, or parasitical diseases, typically viral or bacterial disease. Viral diseases to be treated may be mediated by a poliovirus, adenovirus, coronavirus or herpesvirus.

The compositions may be formulated as a nasal spray, injectable serum, dermal patch, eyedrops and/or for oral administration.

### SUMMARY OF THE FIGURES

FIG. 1 shows an example method of preparing extracts for use in accordance with embodiments of the invention.

FIG. 2 shows the results of a virus inactivation test using extracts according to the invention using as raw material inoculated zabrus from hives inoculated with human coronavirus OC43. FIGS. 2A, B and C, respectively show an A549 culture infected with HCoV-OC43 positive control; a culture not infected with virus (negative control); and a culture infected with HCoV-OC43 virus mixed with the inoculated zabrus preparation in isotonic marine plasma.

### DETAILED DESCRIPTION

The present disclosure provides therapeutic compositions which comprise a bee-derived component and marine plasma; and/or therapeutic compositions which comprise a bee-derived component derived from zabrus. The compositions aim to improve immunogenicity and aid in the induction of desired immunological responses against infectious diseases from pathogens, such as viruses including influenza, poliovirus and coronavirus, such as SARS-CoV-2, as well as other microbial parasites, bacteria and fungi. As such, the compositions described herein may be considered as antimicrobial compositions, anti-viral compositions, antibacterial compositions, and/or as compositions with anti-viral and/or antibacterial properties. They may be used to treat, relieve the symptoms of, or prophylactically act as a preventative against pathogen-induced disease. The compositions may be used to aid in the induction of a lasting ability for the immune system of the recipient to combat a particular pathogen, for example, as an aid to a vaccine response.

### Bees and Bee-Derived Components

Products derived from rearing of bees and bee hives, also referred to as products of apiculture, have been used throughout human history for their antimicrobial and other medicinal properties using components such as honey, royal jelly, propolis and pollen (Denisow, Denisow-Pietrzyk. “Biological and therapeutic properties of bee pollen: a review”. J Sci Food Agric. 2016; Ahmad et al “New Insights into the Biological and Pharmaceutical Properties of Royal Jelly” Int J Mol Sci. 2020; RU2376978C2). The compositions of the present invention comprise one or more bee-derived components—that is, substances made by or obtained from bees, their hives, or otherwise from their activity, or components which are produced from such substances. Given the widespread agricultural use of bees, bee-derived components are easily transportable to laboratories and other production facilities, and can be cost-effective for large scale production, and easily scaled.

Biologically active substances are naturally present in bee products, and include enzymes, vitamins, essential amino acids and essential fatty acids. It is thought that these may have radioprotective, cardiotropic and immunostimulant effects even in low concentration, as well as beneficial impacts on disturbed metabolic cycles.

Honey matured in honeycomb cells is sealed with wax caps or lids, which beekeepers cut off before pumping out honey. ‘Zabrus’ is a term that refers to these caps or upper lids that contain high quality wax, pollen, propolis, honey, enzymes, and other components. For example, the enzymes include lysozyme, which bees add to the caps to protect honey within from bacterial or other microbial contamination. Due to the lower specific gravity in comparison with other components of honey, lysozyme is predominantly concentrated in the upper part of the honey-containing cell, which, while not wishing to be bound by theory, may explain the higher concentration of lysozyme in zabrus, compared to pumped or sold honey, or other parts of the honeycomb. Organic acids, free fatty acids, minerals, vitamins such as carotene, essential oils, proteins, fats, balsams and resins have also been identified in zabrus. Beekeepers themselves do not always consider it necessary to sell zabrus, with some even discarding this substance, as a waste product, and as a result, the present invention also provides a use for this substance.

Zabrus has natural histamine-blocking properties, meaning that it can be used even by individuals with allergies to bees or bee products. Zabrus has previously been used to enhance immunity in individuals with respiratory diseases, diseases of the nasopharynx, digestive diseases and oral cavity diseases. Furthermore, when the active bee-derived component used in the composition is isolated, such as if lysozyme is extracted from bee products, potential allergens such as pollen or venom can be excluded.

The decline of bee populations is of great significance to ecosystems around the world, and in particular, with respect to human activity, in relation to the pollination of plants, such as food crops. One of the reasons for the decline is thought to be a reduction in the ability of bees to ward off infections from colonies. With these considerations in mind, compositions as described herein may be sourced with specific beehive fortification techniques and/or specific feed mentioned below.

For optimal production of the bee-derived components and apicultural products used in the present invention, one or more techniques can be used in the farming of the bees themselves, to protect and care for the bees and the hive and encourage the formation of a resistant bee colony with strong tolerance to most pathogens. In some embodiments, the bees are fed on a specific nectar feed, which comprises farnesol, an organic compound which is a 15-carbon acyclic sesquiterpene alcohol, and which demonstrates antiseptic effects (Derengowski et al. “Antimicrobial effect of farnesol, a Candida albicans quorum sensing molecule, on Paracoccidioides brasiliensis growth and morphogenesis.” Ann Clin Microbiol Antimicrob. 2009). Farnesol is produced by a number of plants and animals and can be added as a supplement directly to a bee feed, or the bees can be raised in an environment with access to feed sources naturally containing farnesol.

In some embodiments, the bee feed comprises or consists essentially of linden nectar, that is, nectar from trees of the Tilia genus, such as Tilia cordata. Linden nectar contains farnesol, as discussed above, and has been used (primarily as linden honey) as a treatment for disorders such as influenza, tonsillitis, and sore throat. This feed can be provided to the bees in any suitable way known in the art.

In some embodiments, the beehives can be treated by fortifying them with a coating. The hive fortification method suitably involves the application of rosin, or a coating comprising rosin. The rosin is obtained from trees. In some embodiments, one or more applications of approximately 30% turpentine and approximately 70% rosin, is used for hive fortification. Such a coating is used to protect wooden beehives from electromagnetic fields and pathogens. The hive fortification technique used enables the bees to build their own wax and also protects the bee from potentially harmful electromagnetic frequencies. In some embodiments, the coating is applied as soon as a hive is placed in a suitable location, such as a field, as the bees need to absorb the material.

As mentioned above, insect immune mechanisms, in particular in bees, may have a greater degree of specificity and longevity than previously thought (Cooper and Eleftherianos, 2017). It is possible to inoculate or expose a bee colony to a particular pathogen, such as a virus, bacterium or parasite. This may mirror the exposure to pathogens that bees encounter in nature, for example as a result of pollination activity. Thus, in some embodiments, the bee colony or hive may be exposed to one or more pathogens before collection of the bee-derived component. It is thought that this will enhance the specificity of the bee-derived component eventually obtained against said pathogen, and/or contribute to increase in production of antiviral components by the colony, thus enhancing the efficacy of the ultimately derived compositions.

Colony inoculation can occur by spraying hives or honeycombs with suspensions of a particular pathogen, typically in a sucrose solution. The pathogen used for inoculation can be a bacterium, a virus, a fungus, a parasite such as a unicellular eukaryote or multicellular parasite, or a immunogenic part or fragment thereof. For example, a suspension of virus can be mixed with a sucrose solution and sprayed onto both sides of all honeycombs. This procedure can be repeated two, three, four, five, or more times, and can take place at intervals of one, two, three, four, five or six days, or can be repeated weekly. The collection of material from the hive for further use can occur after inoculation (or the last inoculation), for example one, two, three, four, five, six, seven, eight, ten, or fourteen days after inoculation. Typically, collection occurs between 11 and 28 days after the first inoculation. Criteria for collection can include sealing of the comb lids, and/or saturation of the lids with immune factors. Preparations of pathogens can be produced by appropriate means known in the art.

In some embodiments, the bee-derived component is zabrus, or is derived from zabrus. In some embodiments, the bee-derived component is or is derived substantially only from zabrus, that is, zabrus is isolated from the bulk of the honeycomb on collection. For example, to isolate zabrus, the caps of the honeycomb may be collected in any suitable way, for example, by cutting with a sharp knife. The zabrus may be treated and/or purified to isolate or concentrate particular components, or to remove other components. For example, the zabrus may be treated to remove wax or other substances with low water solubility (i.e. hydrophobic components), in order to improve water solubility of the resultant product. A diffusion agar method may be used for identification and cation method may be used for extraction of subcomponents of zabrus, for example, lysozyme. Exemplary methods for preparation of suitable extracts are discussed below.

The bee-derived component may be honeycomb, or derived from honeycomb, which may be obtained with or without the attached caps (zabrus). Again, the honeycomb may be treated as described for zabrus in order to prepare a usable extract, typically wherein hydrophobic components have been removed.

In some embodiments, the bee-derived component comprises or is derived from one or more of honey, royal jelly, propolis and pollen.

Bee-derived components themselves derived from raw material as described may have at least a proportion of the hydrophobic components of the raw material removed. For example, the bee-derived components may have 10%, 20%, 30%, 40%, 50%, 60%, 70%, 80%, 90%, 95%, 98%, 99%, or substantially all of the hydrophobic components removed compared to the raw material.

Bee-derived components themselves derived from raw material as described may have at least a proportion of the sugar content of the raw material removed. For example, the bee-derived components may have 10%, 20%, 30%, 40%, 50%, 60%, 70%, 80%, 90%, 95%, 98%, 99%, or substantially all of the sugar content removed compared to the raw material.

Honey possesses numerous biologically active components. Polyphenols in honey are thought to be responsible for antioxidant, antimicrobial and immuno-activating properties. The main antimicrobial activity is towards bacteria, fungi, parasites and viruses. It has been reported that honey stimulates T-lymphocytes in cell culture to multiply, and activates neutrophils. (Al-Waili and Haq “Effect of honey on antibody production against thymus-dependent and thymus independent antigens in primary and secondary immune responses” 2004 Journal of Medicinal Food; Abuharfeil, Al Oran, Abo-Shehada “The effects of bee honey on the proliferative activity of human B and T lymphocytes and activity of phagocytes.” 2008 Food and Agricultural Immunology; Bakr et al “Characteristics of Bioyoghurt Fortified With Fennel Honey.” 2015 Int J Curr Micr App Sci).

Pollen contains polyphenols, vitamins B3, A and E, minerals and sterols. (Rzepecka-Stojko, Anna et al. “Polyphenols from Bee Pollen: Structure, Absorption, Metabolism and Biological Activity.” Molecules (Basel, Switzerland) vol. 20,12 21732-49. 4 Dec. 2015)

Humans use bee pollen as a functional food with many biological properties, the main ones being sport performance, and its antioxidant and anti-microbial effects. In the context of this disclosure, pollen may refer to the plant pollen extract coming from the core of the pollen grain, rather than the whole pollen grain. This may be obtained from beehives.

Royal jelly, a glandular secretion used to feed larvae and queens, is thought to have antiviral effects against Herpes viruses and against Coxsackie viruses. Some studies have suggested immune stimulating activity in animals or cell cultures, including increases in leucocyte counts.
(Wang Bailong et al “Previously Unknown Gamma Globulin in Royal Jelly With Immune Stimulating Properties/Increasing Life Expectancy Of Lab Animals By 30%” 1987)

Propolis, a resinous mixture produced by bees from a mixture of saliva and beeswax with exudate gathered from botanical sources, believed to have antiviral effects. For example, effects of both poplar and Baccharis propolis may have been demonstrated against pathogenic viruses including Adenovirus, Coronavirus, Coxsackie viruses Herpes simplex (HSV-1, HSV-2, Human T-Lymphocyte Virus-(HTLV-1). Quercetin and luteolin, (components of poplar propolis) have antiviral activity against SARS-CoV virus, the pathogen of SARS. Effects of propolis on the protein kinase PAK1 have also been indicated. The abnormal activation of PAK1 is thought to be involved in the pathology of a wide variety of diseases such as cancers, inflammation, viral infection, malaria, and immuno-suppression. Therefore, propolis might be useful for blocking coronavirus-induced fibrosis of lungs and stimulating the immune system. The immune stimulating properties of propolis have been documented (Sforcin, “Propolis and the immune system: a review” Journal of Ethnopharmacology, 2007).

In some embodiments, the bee-derived component is bee venom, which has been shown to possess antiviral activity (Uddin et al “Inhibitory effects of bee venom and its components against viruses in vitro and in vivo.” Journal of Microbiology 2016; Mansour et al “Evaluation of Antiviral Activity of Bee Venom, Phospholipase A-2 (PLA-2) and Propolis against DNA and RNA Virus Models.” IJSRP 2016). The main component, melittin, has many biological properties, including antiviral activity. Bee venom demonstrates activity against Adenovirus, Enterovirus, Herpes Virus (HPV16, 18), HIV, Picornavirus, Influenza A (PR8), Leukaemia Virus Vesicular Stomatitis (VSV), Respiratory Syncytial (RSV), Enterovirus-71 (EV-71) and Coxsackie (H3)-viruses. Immune activating properties have also been demonstrated, which are thought to be due to Phospholipase A264. It has also been proposed that by increasing the immune response bee venom can help the body to fight swine influenza A (H1N1) 87 (PSI). Russian apitherapists have shown that 5 to 6 prophylactic treatments of bee venom can significantly decrease the risk of PSI.

In some embodiments, the bee-derived component comprises or consists of lysozyme, also known as muramidase, which may be derived from zabrus, or from other sources of bee-derived components such as honey. Lysozyme is an antimicrobial enzyme present in many animals that performs a role in the innate immune system. Lysozyme is present in bodily secretions such as mucus, tears, breast milk and saliva, as well as animal-derived components such as egg white. It is present in the lungs, digestive tracts and liver, and is stable at a wide pH range. The antibacterial properties of lysozyme are thought to stem from catalysing the hydrolysis of linkages in peptidoglycan cell walls, with lysis of the bacterium following. However, antimicrobial activity has been reported following mutation of amino acids within the active enzymatic site, suggesting that other mechanisms of action are possible. Some studies have indicated antiviral activity for lysozyme (Ferrari et al “Antiviral Activity of Lysozyme” Nature 1959; Malaczewska et al “Antiviral effects of nisin, lysozyme, lactoferrin and their mixtures against bovine viral diarrhoea virus” BMC Vet Res. 2019). Lysozyme has been added to gastrointestinal treatments for the elderly and to baby formula to aid digestibility. It is used in skincare, to cure and prevent acne. In Japan, lysozyme is used as a prescription to treat headaches, colds and throat infections.

Lysozyme is also thought to have potential effects based on piezoelectric activity (Stapleton et al. “The direct piezoelectric effect in the globular protein lysozyme.” Applied Physics Letters, 2017). Piezoelectricity is the electric charge that accumulates in biological matter such as bone, DNA, various proteins and viruses. This combined with lysozyme's low molecular weight is thought to improve compatibility with cellular ion channels.

Any suitable method known in the art may be used to isolate lysozyme, where this is desired. Lysozyme extraction and purification methods, generally from egg white, have been known for many years (Alderton Ward and Fevold, “Isolation of lysozyme from egg white’, JBC 1944). These may include (a) adsorption of lysozyme on bentonite (a montmorillonite clay), (b) elution of inactive contaminating proteins from the clay by successive washings with phosphate buffer (pH 7 to 8) and 5 percent aqueous pyridine, and (c) elution of the active material with pyridine-sulfuric acid solution at pH 5.0. The eluate may be dialyzed and dried in the frozen state. A white powder can be obtained containing 85 to 90 percent of the lysozyme contained in the egg white.

More recently developed methods are also known (Dekina et al, “Isolation and Purification of Lysozyme from the Hen Egg White” Biotechnologia 2015), for example involving differential heat denaturation of proteins with changing of the medium pH value, followed by neutralization, dialysis and additional purification by gel chromatography. A diffusion agar method or a turbidimetric method, or any other suitable method of lysozyme presence in bee products, for harvesting could be used for determinant.

Other factors which may be present in the bee-derived component include sulfidryl enzymes, and balsams. More generally, the bee-derived component can comprise vitamins, enzymes, essential amino and fatty acids.

Other compounds within bee-derived components which possess antimicrobial and antiviral activity include essential oils, flavonoids, benzoic acid, abscisic acid as well as 10-oxydecenic acids. Without wishing to be bound by theory, it is thought that the remarkable antiviral activity of the compositions described and demonstrated herein is assisted by the presence of several components: phenoxidase (controlled by the prophenoloxidase (proPO) activation system), Lysozyme, defensins, flavonoid compounds, glucose oxidase, methylglyoxal, phenolic acids, hymenoptaecins and apidaecins. Hymenoptaecins and apidaecins are antibacterial polypeptides produced by insects and other animals. Compositions according to the invention can comprise one or more of these components.

### Methods of Preparing Extracts

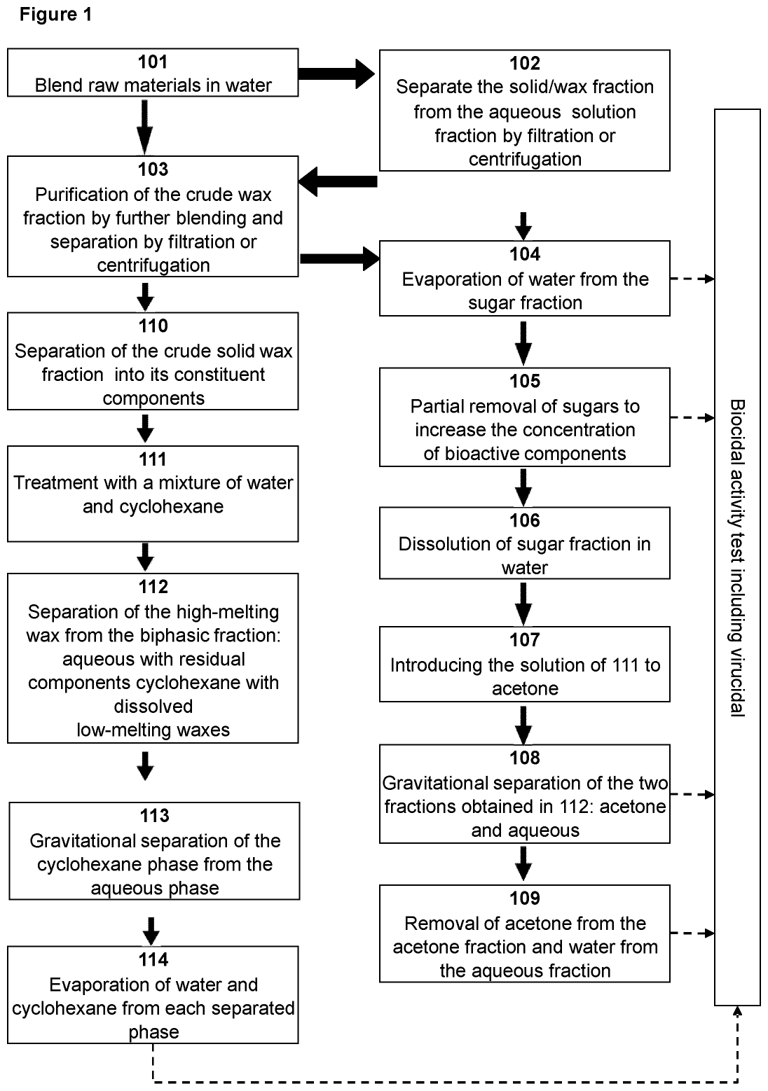

The following describes an example method of preparing extracts for use in accordance with embodiments of the invention, as further exemplified in FIG. 1. Suitably the extracts comprise an aqueous extract comprising a mixture of hydrophilic components obtainable from an apicultural product, such as honeycomb and/or zabrus.

As raw material for the extraction process, honeycombs can be obtained from beehives, which may be treated, inoculated and/or fortified as described elsewhere herein, by any suitable method. Depending on the desired parts to be used, whole honeycombs with sealing caps can be used, or the sealing caps can be removed from the honeycombs by any suitable method, typically using a sharp knife, that is, to isolate zabrus. Sealing caps or pieces thereof can also be used as the material for the following steps. The material may or may not be contaminated with residual honey. The material may be frozen at this stage for ease of transportation.

The material can be crushed by any suitable method or otherwise rendered into small pieces. Subsequently, a suspension in water of the hydrophobic components of the raw materials (mainly waxes and water-insoluble proteins, vitamins or enzymes, essential oils, carotenoids, etc.) is prepared (101). This may be done by dosing the crushed material into a blender, typically a high speed (>30 000 rpm) blender, together with demineralised water or preferably double distilled water. The material is blended, for example for at least 5 minutes, at least 10 minutes, at least 15 minutes, or at least 1 hour.

The suspension/emulsion prepared is then separated (102), for example by filtration, centrifugation and/or evacuation under vacuum, in order to produce a precipitate. Evacuation may be carried out through a sintered-glass plate filter.

The resultant precipitate may be disrupted by blending with additional water as discussed above to form a further suspension/emulsion, and subsequently separated as also discussed above (103). These steps of suspension and separation may be repeated for a total of two, three, four, five or more cycles. The intention is to separate the hydrophilic phase components (the ‘sugar’ phase) from the hydrophobic (or ‘wax’) phase components as completely as possible. Repeating the procedure promotes separation of the water-soluble components from the hydrophobic wax fraction.

Once this is done, the resultant hydrophilic and hydrophobic phases may each be further processed separately to extract desired components, although it is contemplated that only one or the other phase may be processed in some embodiments.

The aqueous fractions (for example, filtrates or supernatants) prepared in the previous steps may be combined at this point. At this stage, the resultant fraction may be concentrated (104), for example by vacuum evaporation, to produce a thick, viscous hydrophilic product/solution. This is understood to comprise honey sugars with an average content of approx. 40% glucose, 40% fructose, 3-4% maltose, and 5% polysaccharides, and a number of water-soluble natural products, including lysozyme, as discussed above.

In some embodiments, a step of precipitating sugars from this product can be included in order to increase concentration of the lysozyme and/or other biocidal substances (105). This can be done by precipitation using polar solvents, typically by introducing part or all of the sugar fraction into dipolar liquids in which the simple sugars are weakly or completely insoluble, for example, acetone, anhydrous ethyl alcohol, or propanol-2, or DMSO, and so forth, with other alcohols considered to also be suitable. Since it is desired to preserve the structure of any protein components, especially lysozyme, solvents which do not denature such components are preferred, particularly acetone.

Accordingly, this precipitation step can take place using a mechanical stirrer containing a polar solvent as described, and introducing the hydrophilic fraction gradually, together with water, in order to produce Solution I (106). A milky suspension is obtained which stratifies into a top solvent phase, and a bottom water phase.

The water is used to extract the lysozyme and other components from the suspension of the sugar fraction in acetone at least partly. Both phases are centrifuged together, after which the acetone solution (Solution II) and aqueous solution (Solution III) become completely separated and are isolated from each other, typically in a separating funnel (108).

The acetone fraction may have the acetone evaporated (109), typically in a vacuum evaporator, to produce an aqueous solution (Solution IV) of simple sugars and other hydrophilic substances and, importantly, the expected lysozyme and/or other components. Previously established data relating to the solubility of chicken lysozyme indicates that this cationic polypeptide of dissolves completely in a mixture of 10 ml acetone and 5.2 ml water.

The hydrophobic wax fraction produced in the earlier suspension and separation steps may also be further processed (110). This may be done by heating with a non-polar solvent such as cyclohexane and filtration, to separate high-melting point waxes and produce a solution of low-melting point waxes in cyclohexane (or equivalent solvent), as well as a water fraction containing the remains of water-soluble substances. For example, part or all of the hydrophobic fraction is heated to melting point (65° C. or higher) together with distilled water and cyclohexane, suitably in a glass vessel. After stirring, the hot mixture is filtered (112), typically by vacuum filtration through sintered glass plates, which may itself be heated, either before use or using an electric heating mantle.

The immiscible liquids in the resultant mixture may be separated (113), for example with a glass separating funnel, typically again at elevated temperature (for example above 70° C.) to avoid solidification. These fractions can be further concentrated, for example by vacuum concentration (114). The separated aqueous fraction (Solution V) can, with or without concentration in a vacuum evaporator, be added to one or more of the fractions isolated in other stages (Solutions III and/or IV) or can treated as a separate fraction comprising bioactive molecules.

Similarly, the cyclohexane fraction can be further purified by adding dropwise to excess acetone, to produce a wax precipitate. The resultant mixture can be separated by vacuum aspiration or centrifuge, with the resultant filtrate being a solution of the impurities in the mixture of cyclohexane and acetone. This can be further separated by distillation to isolate the impurities as a mixture of liquid and solid components.

Further extraction methods may be preferred in various contexts, for example to conform to GMP or other requirements, and/or to ensure the collection of various components. For example, it may be preferred to avoid the use of acetone in the extraction process to avoid loss or damage to various potentially microbicidal components.

Accordingly, a further method of preparing extracts for use in accordance with embodiments of the invention can include some or all of the following steps. All vessels and equipment used in these processes must be sterile cleaned before the extraction process begins.

The raw material is extracted from the hives/honeycomb caps with honey or crushed combs with honey as described above. The extracted raw material is stored at 3-8° C. to avoid growth of moulds and/or gram(+) bacteria.

A specified amount of sterile, distilled/demineralised water is poured into a mixer/blender, suitably with a speed of around 30-35 000 rpm. The previously weighed raw material is then gradually dispensed into the operating mixer. This disintegration process, breaking down the raw material in water is suitably carried out for no more than 4-8 min, depending on the added weight of the raw material.

The suspension obtained in the mixer (typically a cloudy, beige-white liquid) is centrifuged. For example, depending on scale, this can be in a bucket centrifuge, with a rotational speed of >16-20,000 rpm, or in high-speed, flow-through centrifuges of the decanter type. Centrifugation to remove hydrophobic suspensions—mainly waxes—suitably takes no less than 10 minutes. The centrifuged liquid from over the precipitate is decanted into a receptacle such as a glass buffer tank.

The disintegration process is then repeated with the centrifuged hydrophobic precipitate. The mixer/blender is activated, suitably at a speed of >16-20 000 rpm with sterile, distilled/demineralised water. Small portions of the extracted precipitate from the centrifuge precipitate are added to the blender periodically (for example every 5-8 seconds), and the precipitate blending process is continued after the whole mass of precipitate has been introduced, suitably for no longer than 4-5 minutes.

The secondary suspension of hydrophobic substances obtained therein is again centrifuged as above, for example with a rotational speed >16-20 000 rpm. The obtained liquid is added to the receptacle mentioned above. If there is a need for extremely thorough removal of water-soluble bioactive substances from the precipitate, the disintegration and centrifugation steps can be repeated one or more further times.

Optionally, if the fractions of the centrifuged liquid collected in the receptacle show the presence of hydrophobic substances in the liquid (for example demonstrated by a slight opalescence of the liquids forming these fractions) then the liquid can undergo further centrifugation at high speed, for a longer period than previous centrifugation, and/or be filtered, for example through a glass filter/Shotta/type G-4/or membrane filter, as would be conventional to remove such substances from the solution.

The obtained substance is a clear liquid, slightly yellow in color, obtained after centrifugation and possible purification. This can be transferred to a vacuum evaporator for concentration, for example at 38-40° C. at a pressure of around 0.15-0.20 B (15 kPa to 20 kPa) and 120 rpm of the stripping flask. This can be carried out until no further water droplets are discharged from the cooler, or otherwise. The resultant product is typically an extremely viscous, deep red-brown colored substance, to which water or other solvent may be added to extract it from the distillation flask,

This product is soluble in water and aqueous solutions such as e.g. marine plasma, saline solution and so on. As an aside, the solubility of this final product in other solvents such as ethanol, some ethers, etc. is greatly reduced by the phenomenon of precipitation of sugars from it by these solvents.

If storage or transport is required, the concentrated aqueous solution obtained in the distillation flask in small volume is poured into final containers, which are placed either in a water bath at 36-38° C. or under infrared radiators, and the residual water is then driven off by a stream of gaseous nitrogen. This can occur under occasional shaking to remove the solid skin that forms while applying this method. Suitably, the containers with the concentrated aqueous solution can be placed in a thermostat-controlled shaker, which will be realized during the production scale of its obtaining.

Following complete evaporation, the containers can be closed, maintaining a nitrogen atmosphere to aid in storage and prevent the development of microbial colonies. Before dissolution into a product for use, the concentrated extract can be sterilised in any suitable way, for example by microfiltration and/or brief γ-ray irradiation.

As an example of quantities, for 100 g of typical bee-derived raw material, 800 ml of H₂O can be used for the first process of obtaining the suspension. For each of the second and any further stages of disintegration, 160 ml of H₂O can be used.

### Marine Plasma

A large proportion of medical compositions, such as vaccines, are administered in aqueous solution, suspension, or otherwise comprise a large percentage of water, with active ingredients accounting for only a very small proportion of the administered preparation. In some embodiments of the present invention, for example those involving compositions comprising bee-derived components derived from zabrus, standard pharmaceutical vehicles can be used, such as water or saline solution, gels, suspensions, emulsions, and so on.

In aspects of the present invention, a marine plasma is used in place of water, saline or other solvent or vehicle. In the present context, marine plasma refers to a fluid comprising seawater. In some embodiments, marine plasma consists of or consists essentially of seawater. In some embodiments, seawater is diluted with water until it is substantially isotonic with bodily fluids such as blood plasma. Thus, marine plasma comprising seawater diluted to such levels can be referred to as ‘isotonic’ marine plasma, while marine plasma consisting of or consisting essentially of seawater, or of seawater diluted to a lesser extent, can be referred to as ‘hypertonic’ marine plasma. By using marine plasma in lieu of water, it is believed that the use of added ingredients commonly used in medical compositions such as preservatives, for example formaldehyde and others, is not necessary. This can allow for a reduction of the cost in manufacture as well as providing a more natural product, lacking the potentially harmful effects of such chemicals such as allergic reaction and/or toxic reaction. This can allow the compositions to be used more frequently and/or prophylactically than compositions which have such potentially harmful chemicals. Thus, in some embodiments, the composition is substantially free from medical preservatives such as formaldehyde.

Other common components of medical compositions include antibiotics, emulsifiers, stabilisers, adjuvants and acidity regulators. While compositions of the present invention may indeed comprise such components, it is considered that use of marine plasma may allow for one or more of these to be omitted entirely, again with the benefit of reducing costs and potentially harmful effects. Thus, in some embodiments, the composition is substantially free from one or more of antibiotics, preservatives, emulsifiers, stabilisers, adjuvants and/or acidity regulators.

The composition of seawater is similar in many respects to the fluids of the human or mammalian body, in particular, blood plasma, as has been understood since the late 1800s and the work of Rene Quinton. This reduces any negative reaction of the body environment to the marine plasma used in compositions of the present invention. Marine plasma is thought to be the result of a combination of factors, produced by vortex plankton blooms, cyclones of oceanographic and biological phenomena that result from specific conditions of light, temperature, currents and weather patterns. These can provide nourishment and profusion of zooplankton which, in the process of feeding on the marine flora (e.g. phytoplankton), secrete a rich and bio-active fluid or serum. When combined with surrounding seawater, this fluid contains numerous major and trace minerals, plus mineral salts, amino acids, DNA, RNA, antioxidants, polysaccharides, essential fatty acids, vitamins, and phytochemicals, in bio-active form.

Marine plasma as present in compositions described herein, or as obtained in methods of making such compositions, may be produced by a specific extraction process and protocol used to ensure that particular concentrations of components are present and to maintain its integrity. Such protocols include harvesting the seawater used as, or used to make the marine plasma from below plankton blooms, for example between about 10 and about 35 metres below such plankton blooms, typically between about 25 and 35 metres below, and suitably around 30 metres below. Plankton blooms can be detected by satellite surveillance to identify colour changes in affected areas. Accordingly, in some embodiments, marine plasma is seawater harvested below plankton blooms, which can be further treated as described elsewhere herein, and/or can be used in hypertonic or isotonic forms, as discussed above.

Further treatment is carried out after harvesting the seawater, and before or after dilution to the required osmolarity, if applicable. This includes micro-filtering, typically double-cold micro-filtering the seawater, suitably with approximately 0.22-micron filtration. Rigorous controls are in place to ensure its purity. The pharmaceutical production unit complies with the ISO 9001, ISO 14001, FDA's Food Supplements GMPs, GMPs and GMP/NCF Cosmetics (ISO 22716) standards.

Ocean water has an osmolarity (solute concentration) of roughly 3 times that of internal body fluids. In order to achieve an osmolarity suitable for administration to a subject, seawater is typically diluted with water, typically purified, distilled, or reverse osmosis water suitable for therapeutic use, in order to produce marine plasma. Typically, where the marine plasma comprises seawater diluted with water, the final concentration will involve 25% to 30% seawater, and 70% to 75% water, suitably approximately 29% seawater and 71% water.

Other elements which may be present in the composition, which are also derived from the marine environment, include fucoidans and ‘marine peptides’. Fucoidans are thought to have antiviral, neuroprotective, and immune-modulating effects. Marine-derived proteins and bioactive peptides have potential for use as functional ingredients in nutraceuticals and pharmaceuticals due to their effectiveness in both prevention and treatment of diseases. Marine peptides are thought to be involved in the activity of compositions as described herein, including against parasites and to promote antioxidant, antihypertensive, anticoagulant, antiproliferative, anti-human immunodeficiency virus, calcium-binding, anti-obesity and anti-diabetic effects (Ngo et al “Biological Activities of Marine Bioactive Peptides” 2013; Cheung et al. “Marine Peptides: Bioactivities and Applications.” Mar Drugs. 2015).

Typically, the marine plasma used in compositions according to the present invention may have a pH of between approximately 6 and 7. The conductivity may be between approximately 14 and 18 mS/cm. Total dissolved solids may be between approximately 7 and 9 g/l. Salinity may be between approximately 8.5 and 10 g/l.

It has been demonstrated that cell membranes contain proteins that play a role in filtration: minerals and trace elements must be in ionic form to cross cell membranes and reach the cell nucleus to activate our genes. The minerals present in marine plasma as defined herein, and their presence in ionic form, are thought to allow improved takeup into cells as necessary. The minerals present in the marine plasma fulfil a number of roles in body function, and may have beneficial epigenetic effects.

Tables 1 and 2 indicate possible mineral content for the marine plasma used.

TABLE 1
Example contents of isotonic composition

| | |
|---|---|
| Aluminium Al 20.33 ppb | Manganese Mn 33.66 ppb |
| Antimony Sb 0.05 ppb | Mercury Hg ND |
| Arsenic As 8.23 ppb | Molybdenum Mo 5.33 ppb |
| Sulfur S 310.66 ppm | Neodymium Nd ND |
| Barium Ba 1.46 ppb | Niobium Nb 00233 ppb |
| Beryllium Be ND | Nickel Ni 17.76 ppb |
| Bismuth Bi ND | Gold Au ND |
| Boron B 2 ppm | Osmium Os 0.053 ppb |
| Bromine Br 23.66 ppm | Palladium Pd 4.33 ppb |
| Cadmium Cd 0.106 ppb | Silver Ag 0.436 ppb |
| Calcium Ca 134 ppm | Platinum Pt ND |
| Cerium Ce ND | Lead Pb 0.2566 ppb |
| Cesium Cs 0.109 ppb | Potassium K 130.66 ppm |
| Zirconium Zr 0.146 ppb | Praseodymium Pr ND |
| Cobalt Co 0.466 ppb | Rhenium Re ND |
| Copper Cu 1.03 ppb | Rhodium Rh 0.266 ppb |
| Chromium Cr 1.033 ppb | Rubidium Rb 38.33 ppb |
| Dysprosium Dy 0.0033 ppb | Ruthenium Ru ND |
| Erbium Er ND | Samarium Sm ND |
| Scandium Sc ND | Selenium Se 0.533 ppb |
| Tin Sn 0.6 ppb | Silicon Si 3.333 ppm |
| Strontium Sr 0.133 ppm | Sodium Na 1347.33 ppm |
| Europium Eu ND | Thallium Tl 0.00566 ppb |
| Phosphorus P 1.633 ppb | Tantalum Ta 0.0133 ppb |
| Gadolinium Gd ND | Tellurium Te ND |
| Gallium Ga 0.133 ppb | Terbium Tb ND |
| Germanium Ge 2.53 ppb | Titanium Ti 5.666 ppb |
| Hafnium Hf 0.023 ppb | Thorium Th ND |
| Iron Fe 12.26 ppb | Thulium Tm ND |
| Holmium Ho ND | Uranium U 1 ppb |
| Indium In ND | Vanadium V 9.66 ppb |
| Iridium Ir ND | Tungsten W 0.12 ppb |
| Lanthanum La 0.0076 ppb | Iodine I 7.66 ppb |
| Lithium Li 62 ppb | Ytterbium Yb ND |
| Lutetium Lu ND | Yttrium Y 0.033 ppb |
| Magnesium Mg 442.33 ppm | Zinc Zn 7.233 ppb |

TABLE 2
Example contents of hypertonic composition

| | |
|---|---|
| Aluminium Al 61 ppb | Manganese Mn 101 ppb |
| Antimony Sb 0.15 ppb | Mercury Hg ND |
| Arsenic As 24.7 ppb | Molybdenum Mo 16 ppb |
| Sulfur S 932 ppm | Neodymium Nd ND |
| Barium Ba 4.4 ppb | Niobium Nb 0,07 ppb |
| Beryllium Be ND | Nickel Ni 53.3 ppb |
| Bismuth Bi ND | Gold Au ND |
| Boron B 6 ppm | Osmium Os 0.16 ppb |
| Bromine Br 71 ppm | Palladium Pd 13 ppb |
| Cadmium Cd 0.32 ppb | Silver Ag* 1.31 ppb |
| Calcium Ca 402 ppm | Platinum Pt* ND |
| Cerium Ce ND | Lead Pb 0.77 ppb |
| Cesium Cs 0.327 ppb | Potassium K 392 ppm |
| Zirconium Zr 0,44 ppb | Praseodymium Pr ND |
| Cobalt Co 1.4 ppb | Rhenium Re ND |
| Copper Cu 13.8 ppb | Rhodium Rh 0.80 ppb |
| Chromium Cr 3.1 ppb | Rubidium Rb 115 ppb |
| Dysprosium Dy 0.010 ppb | Ruthenium Ru ND |
| Erbium Er ND | Samarium Sm ND |
| Scandium Sc ND | Selenium Se 1.6 ppb |
| Tin Sn 1.8 ppb | Silicon Si 10 ppm |
| Strontium Sr 9.4 ppm | Sodium Na 4042 ppm |
| Europium Eu ND | Thallium Tl 0.017 ppb |
| Phosphorus P 4.9 ppb | Tantalum Ta 0.04 ppb |
| Gadolinium Gd ND | Tellurium Te ND |
| Gallium Ga 0.40 ppb | Terbium Tb ND |
| Germanium Ge 7.6 ppb | Titanium Ti* 17 ppb |
| Hafnium Hf 0.07 ppb | Thorium Th ND |
| Iron Fe 36.8 ppb | Thulium Tm ND |
| Holmium Ho ND | Uranium U 3 ppb |
| Indium In ND | Vanadium V 29 ppb |
| Iridium Ir ND | Tungsten W 0.36 ppb |
| Lanthanum La 0.023 ppb | Iodine I 23 ppb |
| Lithium Li 184 ppb | Ytterbium Yb ND |
| Lutetium Lu ND | Yttrium Y 0.10 ppb |
| Magnesium Mg 1327 ppm | Zinc Zn 21.7 ppb |

Further advantages of the use of marine plasma in the present invention include antimicrobial, and in particular antiviral properties. For example, butylated hydroxytoluene (BHT) is an organic antioxidant, naturally present in marine plasma in trace amounts, in particular if isolated below plankton blooms as discussed above, as it is produced by phytoplankton (Babu and Wu, “Production of Natural Butylated Hydroxytoluene as an Antioxidant by Freshwater Phytoplankton”, J Phycol. 2008). Research shows that small amounts of BHT can knock out flareups of lipid enveloped viruses such as Herpes and Cytomegalovirus (CMV) (Freeman et al “Treatment of recurrent herpes simplex labialis with topical butylated hydroxytoluene” Clin Pharmacol Ther. 1985; Kim et al “Inactivation of cytomegalovirus and Semliki Forest virus by butylated hydroxytoluene”. The Journal of Infectious Diseases 1978). Lipid-enveloped viruses include SARS-CoV-2, the agent responsible for the Covid-19 pandemic. Thus, marine plasma and compositions comprising it, as discussed herein, may comprise BHT, and this may explain some of the antiviral activity demonstrated. BHT has also demonstrated other properties against a range of conditions such as cardiovascular disease, cancer, and brain damage and has also been suggested to slow aging (Björkhem et al. “The antioxidant butylated hydroxytoluene protects against atherosclerosis.” Arterioscler Thromb. 1991; Hocman “Chemoprevention of cancer: phenolic antioxidants (BHT, BHA).” Int J Biochem. 1988; Crews et al “BHT blocks NF-kappaB activation and ethanol-induced brain damage.” Alcohol Clin Exp Res. 2006; Harman “Free radical theory of aging: effect of free radical reaction inhibitors on the mortality rate of male LAF1 mice” J Gerontology. 1968).

Further components which may be present in marine plasma include a naturally high amount of silica. The naturally high silica content in marine plasma yields antiviral properties, and research shows that it can make viruses less efficient at infecting other organisms and can even inactivate viruses when it surrounds them (Laidler et al. “Reversible Inactivation and Desiccation Tolerance of Silicified Viruses”, J Virol 2013; https://www.nature.com/scitable/blog/viruses101/viruses_coated_in_silica_exhibit/; Maruyama et al. “Possibility for controlling global warming by launching nanoparticles into the stratosphere” Journal of Thermal Science and Technology 2015; Martin K R “The chemistry of silica and its potential health benefits.” J Nutr Health Aging. 2007; Pati et al “Nanoparticle Vaccines Against Infectious Diseases” Front. Immunol 2018).

Silica nanoparticles have been shown to have adjuvant activity (Skrastina et al, “Silica Nanoparticles as the Adjuvant for the Immunisation of Mice Using Hepatitis B Core Virus-Like Particles” PLOS One 2014), which may underlie the immune enhancing effect of the compositions described herein. Again, this adjuvant effect stemming from naturally included constituents means that in some embodiments additional adjuvants, such as aluminium salts, may be excluded from the compositions. This has the advantage of reducing any potentially toxic effects of added adjuvants. However, in some embodiments further adjuvants may be added.

### Formulation and Administration

Compositions according to the invention can be formulated and administered in any suitable way. In particular, it is envisioned that compositions may be formulated as a nasal spray, injectable serum, dermal patch, eyedrops and/or as an oral dose. Accordingly, administration may be intranasal, intramuscular, intravenous, intraarterial, subcutaneous, intraperitoneal, topical (including to the surface of the eye), transmucosal, and/or oral, as appropriate.

The composition of bee-derived component with marine plasma may be as an aqueous solution, as any suitable emulsion (for example oil-in-water, water-in-oil, micro-emulsion, multiple emulsion, nano-emulsion) or as an aqueous gel. The bee-derived component may be dissolved, suspended, emulsified, or otherwise carried by the marine plasma. Similar formulations can be used in embodiments where marine plasma is not used.

Information about particular formulations and routes of administration, and the considerations each involves may be found below.

Nasal Sprays. The absorption of the nasal spray is influenced by the residence time on the epithelial tissue in the mucosa. With this method of administration, mucociliary clearance can inhibit the delivery of the formulation to the absorption site. Waxes and oils from the bee products can be added to increase the viscosity, and reduce clearance. If mucociliary clearance increases, the absorption of the product decreases. Otherwise, nasal spray formulations can be developed by suitable means as known in the art. Such formulations have particular utility against microbes which gain access via the respiratory system and associated tissues. Deposition of active ingredients in the anterior and posterior region of the nose affects the active ingredient absorption of the nasal formulation. For example, the SARS-CoV-2 virus is thought to gain cell entry mainly through ACE2 receptors. The nasal cavity (and conjunctival mucosa to which it is anatomically linked) has one of the highest levels of ACE2 receptors in the human body, which makes the nasal mucosa the most important way of coronavirus transmission. The tear film (fluid layer of the eye) contains many antimicrobial, antiviral compounds one of which is lysozyme that works continuously to facilitate the removal of viral pathogens. The nasal spray may offer a boost of lysozyme material (in embodiments where lysozyme is comprised), reaching both the nose and eyes, to replenish the tear film. As a result, the nasal spray (and other formulations) may be used prophylactically as a viral ease/protection formulation.

Dermal Patch. Patches can be used such that the composition can be absorbed through the skin. Such patches may comprise a backing layer that serves as the outer surface of the patch during use, a reservoir layer comprising a composition as described herein, an adhesive for affixing the patch to the subject's skin, and a release liner, which upon removal exposes the reservoir layer and adhesive. The adhesive may suitably be an organic adhesive, and may be comprised within the reservoir layer and/or the composition itself. The reservoir layer may further comprise a permeation enhancer to aid in transdermal absorption. The permeation enhancer may comprise bee venom (as the bee-derived component or in addition to another), or one or more of the components thereof, such as melittin. Melittin is a peptide and a major component of bee venom. It induces membrane permeabilization and lyses cells. Bee venom also possesses biologically active amines such as histamine, epinephrine, dopamine, norepinephrine and enzymes such as phospholipase A2, hyaluronidase, acid phosphomonoesterase, lysophospholipase. Other components of bee venom include lipids, carbohydrates and free amino acids. In a patch formulation, it may be particularly advantageous to formulate the composition as an aqueous gel, colloidal gel or hydrogel, by any suitable means, such that it may be more easily comprised within a patch and held against the skin. Thus, the composition may further include hydrophilic polymer chains.

Injectable composition. As mentioned, compositions may be formulated for injection, which may be intramuscular, intravenous, intraarterial, subcutaneous, or intraperitoneal, suitably intravenous. Typically, such compositions may be formulated in a ratio of 0.5 ml marine plasma or other vehicle, to a thousandth of a gram of bee-derived component, for example, lysozyme. Other bee-derived components may be present if needed, in microgram quantities.

Oral composition. Compositions for oral administration are similar to the injectable form described above. However, as the oral mucosal epithelium is the major site for most pathogens and viruses, more of the formulation can be used. This can be achieved by increasing the concentration of bee-derived component, and/or the inclusion of further bee-derived components or marine products as described elsewhere herein. Oral compositions may be formulated in liquid, gel or capsule form, or in any suitable way. In particular, compositions as described herein can be delivered through food, for example by adding to food, drink, or other nutritional products.

Topical ocular compositions. Compositions as described herein may be formulated to be delivered by application to the surface of the eye (such as by eyedrops) in both humans and animals. Intraocular vaccines in both humans and animals hold the disadvantage of contributing only to local immunity as well as posing a risk of cross reactivity among other reactions and potential side effects. Lysozymes that are naturally occurring in the lacrimal glands and the nasal mucus of both humans and animals are also the main active part of certain embodiments of the compositions described herein, making the compound non-alien to the vaccinated subject.

Additional possibilities for formulation or administration are also contemplated.

### Uses and Efficacy

As described above, both bee-derived components and marine plasma are thought to possess diverse antimicrobial activity, including antibacterial, antiviral, antifungal and antiparasitic activity. The combination of the bee-derived component and marine plasma is particularly advantageous for a number of reasons. The combination of the antimicrobial effects from the components leads to overlapping coverage of multiple potential microbial targets. In addition, if the bee-derived component is selected or produced for efficacy against a specific microbial target, as described elsewhere herein, the combination of specific activity from the bee-derived component and the general activity of the marine plasma can allow for action against a specific target without sacrificing general immune supporting effects. Further, as described, the use of marine plasma instead of an inert solvent or carrier (such as water or saline solution) as is typical in such preparations allows for antimicrobial activity to be provided even in parts of the composition which would usually possess none.

Therefore, the compositions as described herein, or produced by the described methods, are, and can be used as, antimicrobial compositions, antiviral compositions, antibacterial compositions, antifungal compositions, and/or antiparasitic compositions.

As a result, methods of treatment or prevention of various conditions are possible using these compositions. A method of treating a bacterial, viral, fungal, or parasitic disease, typically a viral or bacterial disease, may comprise administering a therapeutically effective amount of the compositions described or produced by the methods described herein, to a subject in need thereof. The compositions may be formulated as discussed above (for example as a nasal spray, injectable serum, dermal patch, eyedrops and/or an oral formulation), and/or administration can be by one or more of the methods as described above.

Methods of treatment of disease may occur through one or more different ways. The antimicrobial effect of the components of the compositions, as detailed above, may have direct action against the pathogens causing disease. The immunogenic or immune-stimulating activity of the components of the compositions, as also described above, may also boost or support the immune system in combatting the disease. Compositions as described herein may also act to relieve the symptoms of various diseases, for example coughs, sore throats, rhinitis, rashes, and other symptoms, and in such a way act as an ease, for example, as a viral ease.

It is also thought that the compositions described and produced by methods described herein may be used in methods of preventing disease, for example bacterial, viral, fungal, or parasitic disease, typically viral or bacterial disease. Again, such methods may comprise administering a therapeutically effective amount of the compositions described or produced by the methods described herein, to a subject in need thereof. The compositions may be formulated as discussed above (for example as a nasal spray, injectable serum, dermal patch, eyedrops and/or an oral formulation), and/or administration can be by one or more of the methods as described above.

Methods of preventing disease may similarly occur in one or more different ways. The antimicrobial effect of the components of the compositions, as detailed above, may have direct action against the pathogens causing disease, killing or deactivating such pathogens before they can enter the body, or reducing the infectious dose such that it cannot establish itself. Such prophylactic activity may also act by preventing ingress of pathogens into the body by the provision of a physical barrier, for instance a protective layer covering mucous membranes and ocular tear film. For example, when administered as a nasal spray or eyedrop, the compositions can prevent entry of pathogens which access the body through the associated mucosa or the respiratory systems by blocking them physically, or by killing or deactivating the pathogens. In such a way, the compositions as described herein may be particularly effective in the prevention and/or treatment of respiratory disease, such as influenza, SARS-CoV and SARS-CoV-2, and others. The immunogenic or immune-stimulating activity of the components of the compositions, as also described above, may also boost or support the immune system of a subject to which they are administrated, such that the compositions may be used as an immune support or booster.

The provision of lysozyme and other bioactive factors may act to replenish the limited amount of bioactive agents produced by the human or animal body, for example at the tear duct, the oral cavity and the tracheobronchial airways of the lung. This can have the effect of reducing viral load, and/or the number of particles taken up, which can aid in prevention of infection. Even in infected individuals, this can reduce the amount of penetrating virus, while stimulating immune response, thereby allowing development of immunity while reducing symptoms, alleviating the course of disease and enhancing the efficacy of the body's defences.

More specifically, it is envisioned that compositions as described or produced by methods described herein may be used to improve an ongoing immune response against a particular pathogen in a subject, thus providing the subject with a lasting immunity against that pathogen, similar to a vaccine response. As described above, it is possible to inoculate a colony of bees with a particular pathogen, in order to induce an immune response against such a target. If the bee-derived component obtained from bees which have been so treated possesses specific activity against that pathogen, it is contemplated that a composition comprising such a bee-derived component can be used specifically against that pathogen directly, and/or to aid in the induction of an immune response against that pathogen in a subject via administration to that subject.

It is also thought that the compositions of the present invention can improve or ameliorate certain common problems with oral vaccine. It is agreed upon that an oral vaccine failure is due mainly to oral tolerance mainly. Oral vaccination in particular is challenging due to many factors including the harsh gastro-intestinal environment meaning it is difficult for delivered antigen to survive, and mechanisms of oral tolerance which reduce immune response to ingested matter. Compositions of the present invention aim to bypass the challenging problems of oral route administration that generally occur. Oral vaccination in this way provides the possibility of stimulating two immune responses; the humoral and cellular immune response at systemic and mucosal sites. The naturally occurring peptides in marine plasma can induce and improve oral tolerance, therefore upping the chances of oral vaccine success.

The potential pathogens and associated diseases which the described compositions can be used to treat, relieve and/or protect against include bacterial, viral, fungal, or parasitic diseases. For example, these include viral diseases caused by agents such as influenza, coronavirus such as SARS-CoV-2 and variants thereof, and rhinovirus. Bacterial targets may include Salmonella enteritidis, Escherichia coli, Staphylococcus aureus, and others.

Moreover, due to differences in geographical distribution of pathogens, and the ability of bees to encounter pathogens in their surroundings, the present invention can easily provide customised products for every region of the world, by situating the source hives in particular locations and/or providing them with customised pathogen compositions.

While some of the mechanisms of antimicrobial action of certain components within the compositions as described herein are known, such as the enzymatic action of lysozyme on bacterial cell walls, others are not so well understood. It is thought that epigenetic effects may underlie certain of the effects. Discussions of the possible effectors of insect immunity can be found in the art (Chan et al. “The innate immune and systemic response in honeybees to a bacterial pathogen, Paenibacillus larvae.” BMC Genomics. 2009).

The well documented antibacterial effect of lysozyme is of help in protection against secondary infection that occurs after viral exposure. Due to the stability of lysozyme, it can safely be administered via oral dose as it can survive the acidic environment of the stomach.

Compositions as described herein can be used on human subjects, but can also be used on non-human animals, such as non-human mammals, and domesticated animals kept for meat or other purposes. In particular, it is considered that poultry, especially domesticated poultry, may be a target for compositions as described herein.

In the last decade, the financial losses caused by the major epidemic diseases of poultry have affected tremendously both the commercial sectors and the public ones. Poultry diseases also have the possibility of being transmitted to humans, that is, of being zoonotic, and posing a threat to public health. Infectious diseases in chickens that are contagious to people include viruses (such as Avian influenza, Newcastle disease), bacteria, which may be food-borne (such as Salmonella enteritidis, Escherichia coli) to parasites, fungi, and others.

Livestock health crises affect many parts of the world. For example, in the MENA region, such problems have included H5N8, H9N2, Gumboro, Newcastle virus, Infectious Laryngotracheitis, IB 4-91, and Avian Infectious bronchitis virus strain IB Ma5. Some of these infectious diseases had no developed vaccines at the time, leading to massive livestock incineration programs and to farms being shut down.

An advantage of the present compositions as a prophylactic antimicrobial composition in domesticated animals is that they could take the place of antibiotic usage. Antibiotics run the risk of prompting the evolution of resistant microbial strains, which could be transmitted to humans. Additionally, certain agricultural practices may mean that antibiotic usage is prohibited.

Similarly, a benefit of all the compositions as described herein is that they comprise natural ingredients and can exclude harmful chemicals and minimise side-effects. In particular, this means that such compositions can be used over long periods of time without concerns of a buildup of potentially harmful ingredients. This is especially helpful when used as a prophylactic and/or immune boosting composition, as it can be taken and its benefits can be maintained during daily life, and/or during periods of higher disease risk, such as during winter and epidemics/pandemics. The natural origin of the components of the compositions can also reduce the risk of drug resistance arising in the microbes it is directed against, as well as allowing the circumvention of resistance against standard treatments which may have already arisen. As a result, compositions as described herein may also be used in addition to other treatments, again aided by the low risk of side effects.

### EXAMPLES

Preparation of virus suspensions: For preparation of virus suspension HeLa cells were cultivated in a 175 cm³ flask (NEST SCIENTIFIC Biotechnology, New Jersey, USA) with Dulbeco's Minimum Essential Medium with Earle's BSS and 10% fetal bovine serum (FBS). Poliovirus type 1, LSc 2ab (Picornavirus), non-enveloped RNA virus (stock virus suspension) was added to the monolayer for 1 h at 37° C. with gentle shaking every 15 min. After cells showed cytopathic effect, they were frozen (−80° C.) and defrosted 3 times followed by a low-speed centrifugation (10 min., 1500×g) in order to sediment cell debris. After aliquoting, the virus was titrated and a Median Tissue Culture Infectious Dose (TCID₅₀) of 10-12 was determined. The virus suspension was then distributed into tubes in volumes of 1 ml and given to the hives for inoculation.

6 further viruses were produced in the same way (Adenovirus 5, Adenovirus 36, Coronavirus OC43, Herpes Simplex and Equine Herpes Virus Type 1).

Bee colony management and inoculation: Bee colonies of the species Apis mellifera carnica from the experimental and didactic apiary of the University of Life Sciences in Wroclaw (UPWr) located in Swojec were used for certain experiments, namely, the inoculation of polio virus as discussed below. The colonies were healthy, in good condition, and inhabited two bodies of the hive. For other experiments, colonies of wild bee Apis mellifera mellifera (Augustow line) were used, inhabiting hives made from wood.

After assessing the families in terms of brood abundance, colonies were selected for particular inoculation. During this stage, no treatments with chemicals (e.g. acaricides for the treatment of mites and/or ticks) were carried out in the colonies, which may result in increased robbery between families. The bees had constant access to food during the experiments.

Inoculation of colonies with viral preparations was carried out by double-sided spraying of all honeycombs. 5 ml of virus suspension was mixed with 95 ml of 50% sucrose solution to produce the composition for spraying. For each virus, this was carried out by applications every four days for a total of four times. 9 days after the final application, material was harvested from the colonies for further use.

Material processing: The material isolated from the bee colonies for further processing included the following, processed into separate extracts:
Inoculated combs—Crushed honeycombs without sealing caps (that is, without zabrus as discussed herein), from colonies inoculated with virus as described;
Inoculated zabrus—pieces of sealing caps from colonies inoculated with virus as described, including some contamination with honey and wax;
Non-inoculated combs—Crushed honeycombs without sealing caps (without zabrus), from colonies not inoculated with virus;
Non-inoculated zabrus—pieces of sealing caps from colonies not inoculated with virus.

These materials were each dosed gradually into a blender containing sterile, distilled/demineralised water at over 30 000 rpm for 4-8 minutes to disintegrate the material, producing a milky suspension in water of the hydrophobic components of the raw materials.

The suspension obtained in the mixer (cloudy, beige-white liquid) was transferred to a bucket centrifuge, with a rotational speed of >16-20,000 rpm for 10 minutes. The centrifuged liquid from over the precipitate was decanted into a glass buffer tank.

The disintegration and centrifugation steps were carried out on the centrifuged hydrophobic precipitate at least one more time, at a blender speed of >16-20 000 rpm for 4-5 minutes and the centrifuged liquid transferred again into the glass buffer tank. Where the presence of hydrophobic substances was subsequently evident in the liquid collected in the buffer tank (e.g. a slight opalescence of the liquids forming these fractions), the liquid was subsequently filtered and/or centrifuged as described above.

The obtained clear liquid preparation was evaporated under vacuum at 38-40° C. at a pressure of 0.15-0.20 B and 120 rpm of the stripping flask until the droplets of water discharged from the cooler into the liquid receiver disappeared, resulting in a very thick, viscous hydrophilic product. This was removed with water and subsequently processed for storage where desired by removal of the remaining water under nitrogen flow as described elsewhere herein.

To produce the final extracts, the product as described above was combined with marine plasma, specifically isotonic or hypertonic marine plasma obtained from Quinton Medical, product reference numbers PT200 and PT201 respectively (Laboratoires Quinton International, S.L., Alicante, Spain). The initial solutions have the consistency of honey. They were suspended in marine plasma (hyper- or isotonic). The final concentration produced depends on the initial weight of extract obtained, with concentrations discussed below. For example, concentrations of between about 1 and 3% were prepared by suspending extract of between 0.6 g and 0.9 g in 10 ml of marine plasma.

### Example 1—Evaluation of Effectiveness of Preparations Against Poliovirus Type 1 (PV-1)

The objective of this study was to evaluate the virus-inactivating properties of described extract preparations obtained from hives inoculated or not inoculated with polio virus, against poliovirus type 1, using a quantitative suspension assay according to PN-EN 14476+ A2:2019-08. This standard describes a quantitative suspension test for the evaluation of virucidal activity in the medical area, by mixing one part by volume of test virus suspension, one part by volume of interfering substance and eight parts by volume of disinfectant. At specified contact times aliquots are taken and residual infectivity is determined.

Preparation of test virus suspension: For preparation of test virus suspension, HeLa cells were cultivated in a 175 cm³ flask (NEST SCIENTIFIC Biotechnology, New Jersey, USA) with Dulbeco's Minimum Essential Medium with Earle's BSS and 10% fetal bovine serum (FBS). Poliovirus type 1 (stock virus suspension) was added to the monolayer for 1 h at 37° C. with gentle shaking every 15 min. After cells showed cytopathic effect, they were frozen (−80° C.) and defrosted 3 times followed by a low-speed centrifugation (10 min., 1500×g) in order to sediment cell debris. After aliquoting, test virus suspension was stored in aliquots at −80° C.

Infectivity assay: Infectivity was determined by endpoint titration, transferring 0.1 ml of each dilution into 8 wells of a microtitre plate, beginning with the highest dilution. This was followed by the addition of 0.1 of freshly trypsinized HeLa cells (10-15×10³ cells per well). Microtitre plates were incubated at 37° C. in 5% CO₂ atmosphere. The plate was observed every day (7 days) and the cytopathic effect was read by using an inverted microscope (Axio Observer, Carl Zeiss MicroImaging GmbH). Calculation of the infective dose TCID50/ml was calculated with the method Spearman i Karber with the following formula:

$$- {log}_{10} T C I {D}_{50} = {x}_{0} - 0.5 + \sum \frac{r}{n}$$

where:
x₀=log 10 of the lowest dilution with 100% positive reaction
r=number of positive determinations of lowest dilution step with 100% positive and all higher positive dilution steps
n=number of determinations for each dilution step

Inactivation assay: An investigation for determination of virucidal activity was carried out. The prepared extracts were examined as 0.1% solution. To prepare the extracts for use, 0.690 g of extracts prepared from polio inoculated hives as described above was resuspended in water. Half the resultant suspension was diluted in hypertonic marine plasma and half in isotonic marine plasma to make the extracts for use. Then 800 microliters of the extract was mixed with 100 microliters of virus and 100 microliters of PBS. 50 microliters of this mixture was inoculated into 50 microliters of cell culture. The final amount of extract was 0.1%. Contact time was 60 minutes. For more convenient handling, the volumes in this assay were 0.1 ml test virus suspension, 0.1 ml interfering substance (PBS) and 0.8 ml test product (extract (0.17%)+isotonic marine plasma; or extract (0.17%)+hypertonic marine plasma). Immediately at the end of the contact time, activity of the disinfectant was stopped by dilution to 10⁻¹². Titration of the virus control was performed at contact times 0 min. and 60 min.

Determination of cytotoxicity Determination of cytotoxicity was performed with 1 ml test product, in order to determine the concentrations of the product at which no cytotoxicity was detected. This test is done by making a series of dilutions from 10⁻¹ to 10⁻⁸ or higher dilutions (depending on the virus). These are then inoculated into cell cultures to determine how toxic the test product is and whether the changes in the culture cells caused by the toxicity are similar to those caused by the virus.

Cell sensitivity For the control of cell sensitivity two parts by volume double distilled water were mixed with eight parts by volume of the highest apparently non-cytotoxic dilution of the product in PBS. This mixture was added to a volume of double-concentrated cell suspension. After 1 h at 37° C. the cells were centrifuged and re-suspended in culture medium. Finally, a comparative titration of the virus test suspension was performed on the treated and non-treated (PBS) cells.

Control of efficacy for suppression of disinfectant activity A mixture of the preparation with chilled DMEM+2% FCS was incubated in an ice bath for 30 minutes, a series of dilutions to 10⁻¹² were made. Virus was titrated and compared to the test titration.

Reference virus inactivation test As reference for determination of virucidal activity a 1.4% formaldehyde solution was included. Cytotoxicity of formaldehyde test solution was determined with dilution up to 10⁻⁵.

Verification The following criteria were evaluated:
a. The titre of the test virus suspension allowed the determination of ≥4 log₁₀ reduction.
b. The cytotoxicity of the test product (0.1%) allowed the detection of 4 log₁₀ reduction of the virus titre.
c. The starting concentration (0.1%) of the product showed a reduction in virus titre of more than 4 log₁₀ within 60 min.
d. The comparative titration on treated (1:10000 dilutions of the preparation) and untreated cells showed an acceptable difference on virus (<log₁₀).
e. The inhibition efficiency control of the product was ≤0.5 log.

It was determined that these criteria were met, and so that the test according to PN-EN 14476+A2:2019-08 is valid.

Results The results of the virus inactivation test using extracts using as raw material inoculated zabrus, non-inoculated zabrus and non-inoculated combs were prepared as discussed above and formulated in either isotonic or hypertonic marine plasma. In these examples hypertonic marine plasma was undiluted, where isotonic plasma was diluted by one part into three parts of spring water. Each condition was carried out with 8 cell culture unit replicates.

The results are shown in Table 3. In the table, a score of 0 indicates no viral activity detected, and scores of 1-4 indicated detection of virus presence (degree of cytopathogenic effect/CPE).

Evaluation of product toxicity (not shown) under the same conditions did not demonstrate any noticeable toxicity at any tested dilution. Likewise, formaldehyde control experiments are not shown but demonstrated cytotoxicity at dilution levels 1 and 2 and little or absent viral activity at higher dilution.

Summary The average reduction of polio virus activity was:

| | | |
|---|---|---|
| Inoculated zabrus - isotonic | 5.3125 | log |
| Inoculated zabrus - hypertonic | 5.0 | log |
| Non-inoculated zabrus - isotonic | 4.6875 | log |
| Non-inoculated zabrus - hypertonic | 5.0 | log |
| Non-inoculated combs - isotonic | 3.75 | log |
| Non-inoculated combs - hypertonic | 3.75 | log |

According to the standard, a preparation is considered virucidal if, after the recommended exposure time, the virus titre is reduced by at least 4 log₁₀ (inactivation ≥99.99%). Inoculated zabrus extracts (from hives inoculated with polio virus type 1) in isotonic or hypertonic marine plasma were tested at 0.1%, with exposure time of 60 minutes. After this time, a titre reduction of ≥4 log₁₀ was determined, which is equivalent to a virucidal efficacy of ≥99.99%. Accordingly, the formulation may be considered as a virucidal preparations against non-enveloped polio virus type 1.

Virucidal efficacy tests against polio type 1 virus conducted on non-inoculated zabrus and on non-inoculated combs with polio virus. In both cases, the virucidal efficacy for extracts suspended in hypertonic marine plasma was ≥4 log (≥99.99% virus reduction) and for extracts suspended in isotonic marine plasma was 3.5 log (99.95% virus reduction). Equivalent tests using marine plasma only were carried out, but showed only a slight reduction in viral activity (not shown).

### Example 2—Evaluation of Effectiveness of Preparations Against Equine Herpes Virus Type 1 (EHV-1)

The objective of this study was to evaluate the virus-inactivating properties of inoculated zabrus preparations obtained from hives inoculated with equine herpes virus type 1, against equine herpes virus type 1, using a quantitative suspension assay according to PN-EN 14476+A2:2019-08.

Preparation of test virus suspension was carried out substantially as in Example 1, except using RK-13 cells and a stock virus suspension of equine herpes virus type 1.

The infectivity assay was carried out substantially as in Example 1, except using RK-13 cells.

Inactivation assay Investigation for determination of virucidal activity was followed. Inoculated zabrus preparations was examined as 0.1% solution. Contact time was 30 minutes, temperature 10° C. The volumes in this assay were 0.1 test virus suspension, 0.1 ml interfering substance (PBS) and 0.8 ml test product (inoculated zabrus extract (0.17%) and isotonic marine plasma). Immediately at the end of the contact time, activity of the disinfectant was stopped by dilution to 10⁻⁸.

Determination of cytotoxicity, cell sensitivity, and reference virus inactivation test steps were carried out substantially as in Example 1. The criteria for verification were fulfilled, namely that:
a. The titre of the test virus suspension allowed the determination of ≥4 log₁₀ reduction.
b. The difference in titre between control virus and virus in the inactivation assay after 30 min is between log −0.5 and log −2.5
c. The cytotoxicity of the test product (0.1%) allowed the detection of 4 log₁₀ reduction of the virus titre.
d. The starting concentration (0.1%) of the product showed a reduction in virus titre of more than 4 log₁₀ within 30 min.
e. The comparative titration on treated (1:10000 dilutions of the preparation) and untreated cells showed an acceptable difference on virus (<log 10).

It was determined that these criteria were met, and so that the test according to PN-EN 14675 is valid.

Results The results of the virus inactivation test using extracts using as raw material inoculated zabrus were prepared as discussed above and formulated in isotonic marine plasma. The results are shown in Table 4. In the table, a score of 0 indicates no viral activity detected, and scores of 1-4 indicated detection of virus presence (degree of cytopathogenic effect/CPE). t represents the detection of cytotoxicity.

Evaluation of product toxicity (not shown) under the same conditions did not demonstrate any noticeable toxicity at any tested dilution. Likewise, formaldehyde control experiments are not shown but demonstrated cytotoxicity at dilution levels 1 and 2 and absent viral activity at higher dilution.

Summary According to the standard, a preparation is considered virucidal if, after the recommended exposure time, the virus titre is reduced by at least 4 log₁₀ (inactivation ≥99.99%). Inoculated zabrus extracts (from hives inoculated with equine herpes virus type 1) in isotonic marine plasma were tested at concentration 0.1%. The exposure time was 30 minutes at temperature 10° C. After this time, a titre reduction of ≥4 log₁₀ was determined, which is equivalent to a virucidal efficacy of ≥99.99%. Accordingly, the formulation may be considered as virucidal against enveloped equine herpes virus type 1.

### Example 3—Evaluation of Effectiveness of Preparations Against Adenovirus Type 36 (Adenoviridae)

The objective of this study was to evaluate the virus-inactivating properties of inoculated zabrus preparations obtained from hives inoculated with adenovirus type 36, against adenovirus type 36, using a quantitative suspension assay according to PN-EN 14476+A2:2019-08.

Preparation of test virus suspension was carried out substantially as in Example 1, except using A549 cells and a stock virus suspension of adenovirus type 36. Similarly, the infectivity assay was carried out substantially as in Example 1, except using A549 cells.

The inactivation assay was carried out substantially as in Example 1. Similarly, the determination of cytotoxicity, cell sensitivity, control of efficacy for suppression of disinfectant activity, and reference virus inactivation test steps were carried out substantially as in Example 1.

The verification criteria were evaluated substantially as in Example 1 and the criteria were determined to be met, such that the test according to PN-EN 14476+A2:2019-08 is valid.

Results The results of the virus inactivation test using extracts using as raw material inoculated zabrus from hives inoculated with adenovirus type 36 were prepared as discussed above and formulated in isotonic marine plasma. Each condition was carried out with 8 cell culture unit replicates.

The results are shown in Table 5. In the table, a score of 0 indicates no viral activity detected, and scores of 1-4 indicated detection of virus presence (degree of cytopathogenic effect/CPE). t represents the detection of cytotoxicity.

Evaluation of product toxicity (not shown) under the same conditions did not demonstrate any noticeable toxicity at any tested dilution. Likewise, formaldehyde control experiments are not shown but demonstrated cytotoxicity at dilution levels 1 and 2 and little or absent viral activity at higher dilution.

Summary According to the standard, a preparation is considered virucidal if, after the recommended exposure time, the virus titre is reduced by at least 4 log₁₀ (inactivation ≥99.99%). An inoculated zabrus preparation was tested against adenovirus 36 which is linked to obesity, at concentration 0.1%, with exposure time of 60 minutes. After this time, a titre reduction of ≥4 log₁₀ was determined, which is equivalent to a virucidal efficacy of ≥99.99%. Accordingly, the formulation may be considered as virucidal against non-enveloped Adenovirus type 36.

### Example 4—Evaluation of Effectiveness of Preparations Against Herpes Simplex Type 1 (Herpesviridae)

The objective of this study was to evaluate the virus-inactivating properties of inoculated zabrus preparations obtained from hives inoculated with herpes simplex 1 (HSV 1) virus, against HSV 1, using a quantitative suspension assay according to PN-EN 14476+A2:2019-08.

Preparation of test virus suspension was carried out substantially as in Example 1, except using a stock virus suspension of Herpes simplex virus type 1 (HSV 1). Similarly, the infectivity assay was carried out substantially as in Example 1.

The inactivation assay was carried out substantially as in Example 1. Similarly, the determination of cytotoxicity, cell sensitivity, control of efficacy for suppression of disinfectant activity, and reference virus inactivation test steps were carried out substantially as in Example 1.

The verification criteria were evaluated substantially as in Example 1 and the criteria were determined to be met, such that the test according to PN-EN 14476+A2:2019-08 is valid.

Results The results of the virus inactivation test using extracts using as raw material inoculated zabrus from hives inoculated with HSV 1 were prepared as discussed above and formulated in isotonic marine plasma. Each condition was carried out with 8 cell culture unit replicates. As a control, virucidal efficacy tests against herpes simplex virus type 1 were similarly conducted on isotonic marine plasma without addition of a hive extract.

The results are shown in Table 6. In the table, a score of 0 indicates no viral activity detected, and scores of 1-4 indicated detection of virus presence (degree of cytopathogenic effect/CPE). t represents the detection of cytotoxicity.

Evaluation of product toxicity (not shown) under the same conditions did not demonstrate any noticeable toxicity at any tested dilution. Likewise, formaldehyde control experiments are not shown but demonstrated cytotoxicity at dilution levels 1 and 2 and little or absent viral activity at higher dilution.

Summary According to the standard, a preparation is considered virucidal if, after the recommended exposure time, the virus titre is reduced by at least 4 log₁₀ (inactivation ≥99.99%). An inoculated zabrus preparation was tested against HSV 1, at concentration 0.1%, with exposure time of 60 minutes. After this time, a titre reduction of ≥4 log₁₀ was determined, which is equivalent to a virucidal efficacy of ≥99.99%. Accordingly, the formulation may be considered as virucidal against HSV 1. As a control study, virucidal efficacy tests against herpes simplex virus type 1 were conducted on isotonic marine plasma. The virucidal efficacy was 2.75 log (99.75% virus reduction).

### Example 5—Evaluation of Effectiveness of Preparations Against Influenza Virus A/H1N1

The objective of this study was to evaluate the virus-inactivating properties of inoculated zabrus preparations obtained from hives inoculated with influenza virus A/H1N1, against influenza virus A/H1N1, using a quantitative suspension assay according to PN-EN 14476+A2:2019-08 and embryonated chicken eggs according to a modified ISO 18184 standard.

The PN-EN 14476+A2:2019-08 standard describes a quantitative suspension test for the evaluation of virucidal activity in the medical area (Phase 2/Step 1), mixing one part by volume of test virus suspension, one part by volume of interfering substance and eight parts by volume of disinfectant. At specified contact times aliquots are taken and residual infectivity is determined.

The use of chicken embryos for testing with influenza virus is described in the ISO 18184 standard. Suspension of virus with the tested product is inoculated into the allantoic cavity of 10-day-old embryonated chicken eggs, after the exposure time. Then ECEs are incubated for two days at 35° C. and allantoic fluid is tested using hemagglutination test. Hemagglutination indicates viral replication and thus a lack of virucidal properties of the test substance.

Preparation of test virus suspension was carried out substantially as in Example 1, except using a stock virus suspension of influenza virus A/H1N1.

Preparation of chicken embryos: 10-day-old chicken embryos from Rossa hens were used. Embryos were infected by 0.2 ml of the virus suspension inoculated into the allantoic cavity of the embryonated chicken eggs (ECEs). Eggs were incubated in an incubator at 35° C., for 3 days. After incubation, they were placed in a refrigerator to cool overnight, and then the allantoic fluid was collected. All fluids collected from the eggs were examined by hemagglutination to confirm the replication of influenza virus. Just before the test, the virus-containing allantoic fluids were diluted with phosphate-buffered saline (PBS) (pH 7.2) to give a virus concentration of 107 EID₅₀/0.2 ml.

Preparation of a 0.5% suspension of chicken red blood cells: 4.0 ml of chicken venous blood was collected using a syringe containing 1.0 ml sterilized 2% sodium citrate placed in a tube. The blood was centrifuged at 1,000 g for 10 minutes. The supernatant was then collected and the cells flooded with PBS (5 ml). This was repeated twice, and then 0.5 ml of the cell pellet was suspended in 99.5 ml PBS and the suspension was shaken.

Test procedure (ISO 18184/F7) and PN-EN 14476+A2:2019-08: An investigation for determination of virucidal activity was carried out to PN-EN 14476+A2:2019-08 (EN 5. 5. 2). 0.1% solutions of extracts using as raw material inoculated zabrus from hives inoculated with influenza virus A/H1N1 in isotonic marine plasma were prepared as described above. For more convenient handling, the volumes used in this assay were 0.1 ml test virus suspension, 0.1 ml interfering substance (PBS) and 0.8 ml test product (extract from inoculated zabrus and isotonic marine plasma). Immediately at the end of a contact time, activity of the disinfectant was stopped by cooling.

Inoculation of virus suspension into 10-day-old embryonated chicken eggs according to ISO 18184: After the contact time of 60 minutes, 0.2 ml of the prepared suspension of virus with inoculated zabrus preparation was inoculated into the allantoic cavity of three ECEs. ECEs were incubated for two days at 35° C. After two days of incubation, allantoic fluid was collected from the eggs and placed in tubes as described. Then, a 0.5% suspension of chicken red blood cells was added to the tubes. The tubes were observed for hemagglutination.

Calculation of virus titre: EID₅₀ virus titre was calculated using the Reed and Muench method. 50% EID is calculated on the basis of the cumulative number of infected embryos.

The effectiveness value was calculated based on the following formula:

$$Mv = - lg ( Vb / Va ) = - [ lg ( Vb ) - lg ( Va ) ]$$

Where Mv is the antiviral efficacy value; lg(Vb) represents the EID₅₀ of infected embryonic allantoic fluid without contact with the test sample after a defined contact time; lg(Va) represents the EID₅₀ of infected allantoic fluid from embryos in contact with the sample after a defined contact time.

The results of the examination are shown in Table 7, in which: Virus infection (−): Precipitation of red blood cells is observed, but without condensation. Virus infection (+): condensation of red blood cells is observed.

According to the standard, a preparation is considered virucidal if, after the recommended exposure time, the virus titre is reduced in comparison with the control virus. The observed reduction factor was 6 log. Accordingly, the formulation may be considered to be virucidal against the enveloped influenza virus A/H1N1.

### Example 6—Evaluation of Effectiveness of Preparations Against Human Coronavirus OC43 (HcoV-OC43)

The objective of this study was to evaluate the virus-inactivating properties of inoculated zabrus preparations obtained from hives inoculated with human coronavirus OC43 (HcoV-OC43), against HcoV-OC43, using a quantitative suspension assay according to PN-EN 14476+A2:2019-08.

Preparation of test virus suspension was carried out substantially as in Example 1, except using A549 cells, and a stock virus suspension of Betacoronavirus 1 strain OC43 (ATCC® VR-1558™).

Infectivity assay: Infectivity was determined as endpoint titration, transferring 0.1 ml of each dilution into 8 wells of a microtitre plate, beginning with the highest dilution. This was followed by the addition of 0.1 of freshly trypsinized A549 cells (10-15×10³ cells per well). Due to poor cytopathic effect, the presence of virus was determined by indirect immunofluorescence test. For this, the fluid above the culture was removed and the cells were washed with PBS and then for 10 min. was fixed with 4% PFA solution. The culture was washed twice with PBS. To permeabilize the cell membrane, 3 min. 0.3% Triton X solution was applied followed by PBS wash twice. In the next step, nonspecific binding was applied for 40 min. 3% BSA, followed by primary antibody (Anti-HCoV OC43 monoclonal antibody (CABT-B341), CD Creative Diagnostic) for 60 min. After washing twice with PBS, secondary antibodies (Goat Anti-Mouse IgG H&L (Alexa Fluor® 488) (ab150113), Abcam) were applied for 40 min. The plate was observed under a fluorescence inverted microscope (Axio Observer, Carl Zeiss MicroImaging GmbH). Calculation of infective dose TCID50/ml was calculated with the method Spearman i Karber as in Example 1.

The inactivation assay was carried out substantially as in Example 1, except examining inoculated zabrus extract at 3% solution. Results were read by indirect immunofluorescence as described above.

The determination of cytotoxicity, cell sensitivity, control of efficacy for suppression of disinfectant activity, and reference virus inactivation test steps were carried out substantially as in Example 1. The verification criteria were evaluated substantially as in Example 1 and the criteria were determined to be met, such that the test according to PN-EN 14476+A2:2019-08 is valid.

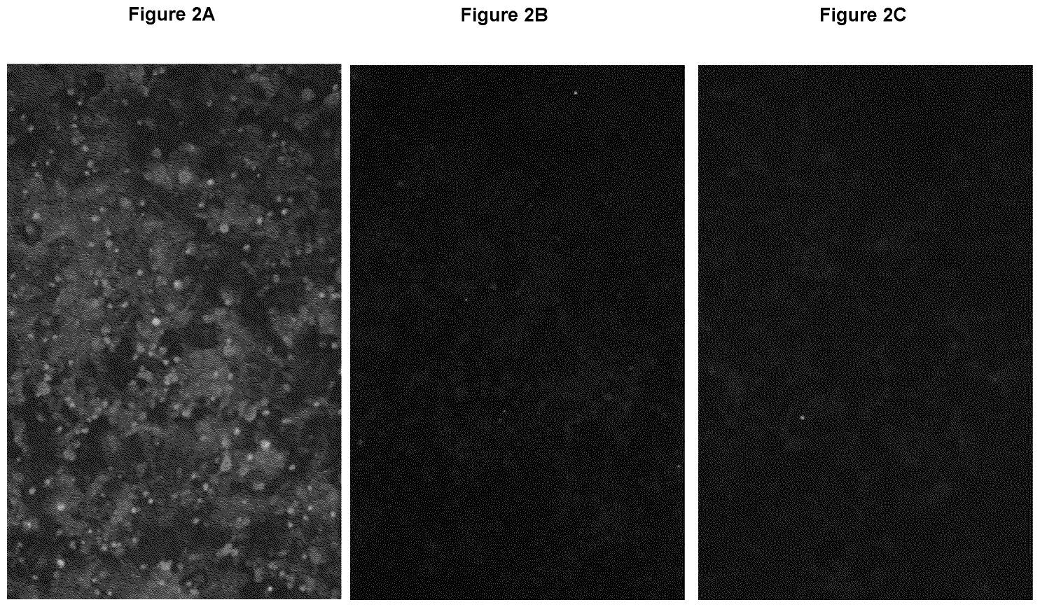

The results of the virus inactivation test using extracts using as raw material inoculated zabrus from hives inoculated with human coronavirus OC43 were prepared as discussed above and formulated in isotonic marine plasma. Each condition was carried out with 8 cell culture unit replicates. Results are shown in FIGS. 2A, B and C, respectively showing an A549 culture infected with HCoV-OC43 positive control, a culture not infected with virus (negative control) and a culture infected with HCoV-OC43 virus mixed with the inoculated zabrus preparation in isotonic marine plasma as described. It can be seen that the viral signal is reduced or absent in the treated culture. Results are also shown in Table 8. In the table, a score of 0 indicates no viral activity detected, and scores of 1-4 indicated detection of virus presence (degree of cytopathogenic effect/CPE). t represents the detection of cytotoxicity.

Summary: According to the standard, a preparation is considered virucidal if, after the recommended exposure time, the virus titre is reduced by at least 4 log₁₀ (inactivation ≥99.99%). An inoculated zabrus preparation was tested against human coronavirus OC43, at concentration 3%, with exposure time of 60 minutes. After this time, a titre reduction of ≥4 log₁₀ was determined, which is equivalent to a virucidal efficacy of ≥99.99%. Accordingly, the formulation may be considered as virucidal against human coronavirus OC43.

### Example 7—Further Evaluation of Effectiveness of Preparations Against Adenovirus Type 36 (Adenoviridae)

The objective of this study was to evaluate the virus-inactivating properties of inoculated zabrus preparations obtained from hives inoculated with adenovirus type 36, against adenovirus type 36, using a quantitative suspension assay according to PN-EN 14476+A2:2019-08.

Preparation of test virus suspension was carried out substantially as in Example 1, except using A549 cells and a stock virus suspension of adenovirus type 36. Similarly, the infectivity assay was carried out substantially as in Example 1, except using A549 cells cultured with Eagle's Minimum Essential Medium with Earle's BSS and 10% fetal bovine serum.

The inactivation assay was carried out substantially as in Example 1, except that the prepared extracts were examined as 10% solution. Similarly, the determination of cytotoxicity, cell sensitivity, control of efficacy for suppression of disinfectant activity, and reference virus inactivation test steps were carried out substantially as in Example 1.

The verification criteria were evaluated substantially as in Example 1 and the criteria were determined to be met, such that the test according to PN-EN 14476+A2:2019-08 is valid.

Results The results of the virus inactivation test using extracts using as raw material inoculated zabrus from hives inoculated with adenovirus type 36 were prepared as discussed above and formulated in isotonic marine plasma. Each condition was carried out with 8 cell culture unit replicates.

The results are shown in Table 9. In the table, a score of 0 indicates no viral activity detected, and scores of 1-4 indicated detection of virus presence (degree of cytopathogenic effect/CPE). t represents the detection of cytotoxicity.

Evaluation of product toxicity (not shown) under the same conditions showed toxicity at dilution levels 1, 2 and 3. Likewise, formaldehyde control experiments are not shown but demonstrated cytotoxicity at dilution levels 1 and 2 and little or absent viral activity at higher dilution.

Summary According to the standard, a preparation is considered virucidal if, after the recommended exposure time, the virus titre is reduced by at least 4 log₁₀ (inactivation ≥99.99%). An inoculated zabrus preparation was tested against adenovirus 36 which is linked to obesity, at concentration 10%, with exposure time of 60 minutes. After this time, a titre reduction of >4 log₁₀ was determined, which is equivalent to a virucidal efficacy of ≥99.99%. Accordingly, the formulation may be considered as virucidal against non-enveloped Adenovirus type 36.

### Example 8—Further Evaluation of Effectiveness of Preparations Against Herpes Simplex Type 1 (Herpesviridae)

The objective of this study was to evaluate the virus-inactivating properties of non-inoculated zabrus preparations obtained from hives not specifically inoculated with any virus, against HSV 1, using a quantitative suspension assay according to PN-EN 14476+A2:2019-08, that is, to investigate the properties of compositions which have been prepared without a step of inoculating the hives against the pathogen against which it is tested.

Preparation of test virus suspension was carried out substantially as in Example 4. Similarly, the infectivity assay was carried out substantially as in Example 4.

Inactivation assay: An investigation for determination of virucidal activity was followed to PN-EN 14476+A2:2019-08 (EN 5.5.2). An extract from caps (non-inoculated zabrus) was prepared from an uninoculated hive. The extract sample was suspended in marine plasma to obtain a 10% suspension. The 10% suspension was the starting preparation for the study and was determined as concentration 0 (C₀). A series of 10-fold dilutions were prepared from C₀:
C₀—10% suspension of the extract
C₁—10% C₀
C₂—1% C₀
C₃—0.1% C₀
C₄—0.01% C₀
   Contact time was 60 minutes. Due to a more convenient handling, the volumes in this assay were 0.1 ml test virus suspension, 0.1 ml interfering substance (PBS) and 0.8 ml test product. Immediately at the end of a contact time, activity of the disinfectant was stopped by dilution to 10⁻¹². Titration of the virus control was performed at contact times 60 min.

The verification criteria were evaluated substantially as in Example 1 and the criteria were determined to be met, such that the test according to PN-EN 14476+A2:2019-08 is valid.

Results The results of the virus inactivation test using extracts using as raw material non-inoculated zabrus were prepared as discussed above and formulated in isotonic marine plasma. Each condition was carried out with 4 cell culture unit replicates. As a control, virucidal efficacy tests against herpes simplex virus type 1 were similarly conducted on isotonic marine plasma without addition of a hive extract.

The results are shown in Table 10. In the table, a score of 0 indicates no viral activity detected, and scores of 1-4 indicated detection of virus presence (degree of cytopathogenic effect/CPE). t represents the detection of cytotoxicity.

Summary According to the standard, a preparation is considered virucidal if, after the recommended exposure time, the virus titre is reduced by at least 4 log₁₀ (inactivation ≥99.99%). A non-inoculated zabrus preparation was tested against HSV 1, at concentrations as described. The exposure time was 60 minutes. After this time, a titer reduction was determined:
C₀—3 log₁₀—inactivation=99.9%
C₁—2 log₁₀—inactivation=99%
C₂—2 log₁₀—inactivation=99%
C₃—0 log₁₀—inactivation=0%
C₄—0 log₁₀—inactivation=0%

The product in C₀ concentrations can be considered virucidal against enveloped herpes simplex virus type 1 at 99.9%. As the dilution increases, the virucidal activity of Beemar decreases. At a C3 and C4 concentration, the preparation does not show virucidal properties.

### Example 9—Further Evaluation of Effectiveness of Filtered Preparations Against Herpes Simplex Type 1 (Herpesviridae)

The objective of this study was to evaluate the virus-inactivating properties of filtered, non-inoculated zabrus preparations obtained from hives not specifically inoculated with any virus, against HSV 1, using a quantitative suspension assay according to PN-EN 14476+A2:2019-08, that is, to investigate the properties of filtered compositions.

Preparation of test virus suspension was carried out substantially as in Example 8. Similarly, the infectivity assay was carried out substantially as in Example 8.

The inactivation assay was carried out substantially as in Example 8, except that the prepared extracts were examined as 10% solution (C₀), with two concentrations prepared:
C₀—10% suspension of the extract
C₁—10% C₀

The verification criteria were evaluated substantially as in Example 8 and the criteria were determined to be met, such that the test according to PN-EN 14476+A2:2019-08 is valid.

Results The results of the virus inactivation test using extracts using as raw material non-inoculated zabrus were prepared as discussed above, formulated in isotonic marine plasma, and filtered. Each condition was carried out with 4 cell culture unit replicates. As a control, virucidal efficacy tests against herpes simplex virus type 1 were similarly conducted on isotonic marine plasma without addition of a hive extract.

The results are shown in Table 11. In the table, a score of 0 indicates no viral activity detected, and scores of 1-4 indicated detection of virus presence (degree of cytopathogenic effect/CPE). t represents the detection of cytotoxicity.

Summary According to the standard, a preparation is considered virucidal if, after the recommended exposure time, the virus titre is reduced by at least 4 log 10 (inactivation ≥99.99%). A filtered, non-inoculated zabrus preparation was tested against HSV 1, at concentrations as described. The exposure time was 60 minutes. After this time, a titer reduction was determined:
C₀—4 log₁₀—inactivation=99.99%
C₁—4 log₁₀—inactivation=99.99%

The product in both C₀ and C₁ concentrations can be considered virucidal against enveloped herpes simplex virus type 1 at 99.99%. This implies that the filtered preparations are more effective than those used unfiltered (see Example 8).

TABLE 3
Testing the virucidal properties of various extracts against poliovirus 1

| | | | Contact | | | | | | | | | | | | |
|---|---|---|---|---|---|---|---|---|---|---|---|---|---|---|---|
| | | | time | Dilution | | | | | | | | | | | |
| | | Conc. | (min) | 1 | 2 | 3 | 4 | 5 | 6 | 7 | 8 | 9 | 10 | 11 | 12 |
| Inoculated | Product | 0.1% | 60 | 4444 | 4444 | 4444 | 3344 | 3344 | 3211 | 2111 | 1101 | 0000 | 0000 | 0000 | 0000 |
| zabrus; | | | | 4444 | 4444 | 4444 | 4434 | 4333 | 2221 | 2111 | 1001 | 0000 | 0000 | 0000 | 0000 |
| isotonic - | Virus | n.d | 0 | 4444 | 4444 | 4444 | 4444 | 4444 | 3443 | 3343 | 3233 | 2233 | 2223 | 2122 | 1111 |
| 1 | control | | | 4444 | 4444 | 4444 | 4444 | 4344 | 3423 | 3323 | 3223 | 3222 | 2222 | 1222 | 0112 |
| | | n.d | 60 | 4444 | 4444 | 4444 | 4444 | 4444 | 3444 | 3344 | 2333 | 2233 | 2223 | 2112 | 1102 |
| | | | | 4444 | 4444 | 4444 | 4444 | 4444 | 3424 | 3323 | 3223 | 2223 | 2213 | 2112 | 1101 |
| Inoculated | Product | 0.1% | 60 | 4444 | 4444 | 4344 | 3343 | 0000 | 0000 | 0000 | 0000 | 0000 | 0000 | 0000 | 0000 |
| zabrus; | | | | 4444 | 4444 | 4434 | 4433 | 0000 | 0000 | 0000 | 0000 | 0000 | 0000 | 0000 | 0000 |
| isotonic - | Virus | n.d | 0 | 4444 | 4444 | 4444 | 4444 | 4444 | 3444 | 3343 | 2233 | 2232 | 2222 | 2111 | 1100 |
| 2 | control | | | 4444 | 4444 | 4444 | 4444 | 4444 | 3423 | 3323 | 3223 | 3222 | 2222 | 1222 | 0112 |
| | | n.d | 60 | 4444 | 4444 | 4444 | 4444 | 4444 | 3444 | 3344 | 2333 | 2233 | 2223 | 1112 | 1101 |
| | | | | 4444 | 4444 | 4444 | 4444 | 4444 | 3424 | 3423 | 3223 | 2223 | 2213 | 2112 | 1101 |
| Inoculated | Product | 0.1% | 60 | 4444 | 3443 | 4322 | 3222 | 0000 | 0000 | 0000 | 0000 | 0000 | 0000 | 0000 | 0000 |
| zabrus; | | | | 4444 | 3344 | 2322 | 1322 | 0000 | 0000 | 0000 | 0000 | 0000 | 0000 | 0000 | 0000 |
| isotonic - | Virus | n.d | 0 | 4444 | 4444 | 4444 | 4444 | 4444 | 3443 | 3343 | 2201 | 0000 | 0000 | 0000 | 0000 |
| 3 | control | | | 4444 | 4444 | 4444 | 4444 | 4344 | 3423 | 2231 | 2221 | 0000 | 0000 | 0000 | 0000 |
| | | n.d | 60 | 4444 | 4444 | 4444 | 4444 | 4444 | 3444 | 3234 | 1211 | 0000 | 0000 | 0000 | 0000 |
| | | | | 4444 | 4444 | 4444 | 4444 | 4444 | 3444 | 3332 | 3132 | 0000 | 0000 | 0000 | 0000 |
| Inoculated | Product | 0.1% | 60 | 4444 | 4444 | 4444 | 3344 | 3333 | 2212 | 1111 | 0001 | 0000 | 0000 | 0000 | 0000 |
| zabrus; | | | | 4444 | 4444 | 4444 | 4444 | 4343 | 2121 | 2111 | 0001 | 0000 | 0000 | 0000 | 0000 |
| Hypertonic - | Virus | n.d | 0 | 4444 | 4444 | 4444 | 4444 | 3444 | 3443 | 4343 | 3232 | 2232 | 2122 | 2121 | 1011 |
| 4 | control | | | 4444 | 4444 | 4444 | 4444 | 4344 | 3423 | 3323 | 3223 | 2222 | 2222 | 1122 | 0112 |
| | | n.d | 60 | 4444 | 4444 | 4444 | 4444 | 4444 | 3444 | 3343 | 3333 | 2233 | 2223 | 2112 | 1102 |
| | | | | 4444 | 4444 | 4444 | 4444 | 4444 | 3424 | 3323 | 3223 | 2223 | 2213 | 2112 | 1101 |
| Inoculated | Product | 0.1% | 60 | 4444 | 4444 | 4344 | 4342 | 3211 | 0000 | 0000 | 0000 | 0000 | 0000 | 0000 | 0000 |
| zabrus; | | | | 4444 | 4444 | 4434 | 3333 | 2121 | 0000 | 0000 | 0000 | 0000 | 0000 | 0000 | 0000 |
| Hypertonic - | Virus | n.d | 0 | 4444 | 4444 | 4444 | 4444 | 3444 | 3444 | 3443 | 2232 | 2232 | 2122 | 2101 | 1100 |
| 5 | control | | | 4444 | 4444 | 4444 | 4444 | 4444 | 3423 | 3323 | 3223 | 2222 | 2222 | 1122 | 0110 |
| | | n.d | 60 | 4444 | 4444 | 4444 | 4444 | 4444 | 3444 | 3334 | 2333 | 2233 | 2223 | 1112 | 1101 |
| | | | | 4444 | 4444 | 4444 | 4444 | 4444 | 3424 | 3423 | 3223 | 2223 | 2212 | 2112 | 1101 |
| Inoculated | Product | 0.1% | 60 | 4444 | 4443 | 3321 | 3211 | 0000 | 0000 | 0000 | 0000 | 0000 | 0000 | 0000 | 0000 |
| zabrus; | | | | 4444 | 3343 | 2322 | 1322 | 0000 | 0000 | 0000 | 0000 | 0000 | 0000 | 0000 | 0000 |
| Hypertonic - | Virus | n.d | 0 | 4444 | 4444 | 4444 | 4444 | 4444 | 3333 | 3322 | 2101 | 0000 | 0000 | 0000 | 0000 |
| 6 | control | | | 4444 | 4444 | 4444 | 4444 | 4344 | 3323 | 2231 | 2221 | 0000 | 0000 | 0000 | 000 |
| | | n.d | 60 | 4444 | 4444 | 4444 | 4444 | 4434 | 3433 | 3233 | 1211 | 0000 | 0000 | 0000 | 0000 |
| | | | | 4444 | 4444 | 4444 | 4444 | 4444 | 3443 | 3332 | 3132 | 0000 | 0000 | 0000 | 0000 |
| Non- | Product | 0.1% | 60 | 4444 | 4444 | 4444 | 3343 | 3223 | 2200 | 1100 | 1100 | 1100 | 0000 | 0000 | 0000 |
| inoculated | | | | 4444 | 4444 | 4444 | 4444 | 4343 | 0210 | 0110 | 0110 | 0110 | 0000 | 0000 | 0000 |
| zabrus; | Virus | n.d | 0 | 4444 | 4444 | 4444 | 4444 | 3444 | 3443 | 4443 | 3232 | 2222 | 2111 | 2111 | 1111 |
| isotonic | control | | | 4444 | 4444 | 4444 | 4444 | 4344 | 3423 | 3323 | 3223 | 2222 | 2122 | 1122 | 0112 |
| | | n.d | 60 | 4444 | 4444 | 4444 | 4444 | 4444 | 3344 | 3343 | 3333 | 2223 | 2223 | 2112 | 1002 |
| | | | | 4444 | 4444 | 4444 | 4444 | 4444 | 3424 | 3323 | 3223 | 2223 | 2213 | 2112 | 1101 |
| Non- | Product | 0.1% | 60 | 4444 | 4444 | 4444 | 3343 | 3222 | 1110 | 1110 | 0110 | 0000 | 0000 | 0000 | 0000 |
| inoculated | | | | 4444 | 4444 | 4444 | 4334 | 3333 | 1110 | 0100 | 0000 | 0000 | 0000 | 0000 | 0000 |
| zabrus; | Virus | n.d | 0 | 4444 | 4444 | 4444 | 4444 | 3444 | 3443 | 4443 | 3232 | 2222 | 2111 | 2111 | 1111 |
| hypertonic | control | | | 4444 | 4444 | 4444 | 4444 | 4344 | 3423 | 3323 | 3223 | 2222 | 2122 | 1122 | 0112 |
| | | n.d | 60 | 4444 | 4444 | 4444 | 4444 | 4444 | 3344 | 3343 | 3333 | 2223 | 2223 | 2112 | 1002 |
| | | | | 4444 | 4444 | 4444 | 4444 | 4444 | 3424 | 3323 | 3223 | 2223 | 2213 | 2112 | 1101 |
| Non- | Product | 0.1% | 60 | 4444 | 4444 | 4444 | 3443 | 3233 | 2222 | 2122 | 0021 | 0011 | 0000 | 0000 | 0000 |
| inoculated | | | | 4444 | 4444 | 4444 | 4334 | 3333 | 2122 | 2121 | 2100 | 1100 | 0000 | 0000 | 0000 |
| combs; | Virus | n.d | 0 | 4444 | 4444 | 4444 | 4444 | 3444 | 3443 | 4443 | 3232 | 2222 | 2111 | 2111 | 1111 |
| isotonic | control | | | 4444 | 4444 | 4444 | 4444 | 4344 | 3423 | 3323 | 3223 | 2222 | 2122 | 1122 | 0112 |
| | | n.d | 60 | 4444 | 4444 | 4444 | 4444 | 4444 | 3344 | 3343 | 3333 | 2223 | 2223 | 2112 | 1002 |
| | | | | 4444 | 4444 | 4444 | 4444 | 4444 | 3424 | 3323 | 3223 | 2223 | 2213 | 2112 | 1101 |
| Non- | Product | 0.1% | 60 | 4444 | 4444 | 3444 | 3343 | 2233 | 2222 | 2132 | 2111 | 0000 | 0000 | 0000 | 0000 |
| inoculated | | | | 4444 | 4444 | 4444 | 4334 | 2333 | 2122 | 2121 | 2111 | 0000 | 0000 | 0000 | 0000 |
| combs; | Virus | n.d | 0 | 4444 | 4444 | 4444 | 4444 | 3444 | 3443 | 4443 | 3232 | 2222 | 2111 | 2111 | 1111 |
| hypertonic | control | | | 4444 | 4444 | 4444 | 4444 | 4344 | 3423 | 3323 | 3223 | 2222 | 2122 | 1122 | 0112 |
| | | n.d | 60 | 4444 | 4444 | 4444 | 4444 | 4444 | 3344 | 3343 | 3333 | 2223 | 2223 | 2112 | 1002 |
| | | | | 4444 | 4444 | 4444 | 4444 | 4444 | 3424 | 3323 | 3223 | 2223 | 2213 | 2112 | 1101 |

TABLE 4
Testing the virucidal properties of extracts against equine herpes virus type 1

| | | | Contact | | | | | | | | | | | | |
|---|---|---|---|---|---|---|---|---|---|---|---|---|---|---|---|
| | | | time | Dilution | | | | | | | | | | | |
| | | Conc. | (min) | 1 | 2 | 3 | 4 | 5 | 6 | 7 | 8 | 9 | 10 | 11 | 12 |
| Inoculated | Product | 0.1% | 60 | tttt | 0000 | 0000 | 0000 | 0000 | 0000 | 0000 | 0000 | 0000 | 0000 | 0000 | 0000 |
| zabrus; | | | | tttt | 0000 | 0000 | 0000 | 0000 | 0000 | 0000 | 0000 | 0000 | 0000 | 0000 | 0000 |
| isotonic | Virus | n.d | 0 | 4444 | 4444 | 4444 | 4444 | 4444 | 3443 | 3333 | 2233 | 2222 | 2222 | 2121 | 1101 |
| | control | | | 4444 | 4444 | 4444 | 4444 | 4444 | 3233 | 3323 | 3223 | 3222 | 2222 | 1211 | 2111 |
| | | n.d | 60 | 4444 | 4444 | 4444 | 4444 | 4444 | 3443 | 3344 | 3333 | 2233 | 2221 | 2212 | 2120 |
| | | | | 4444 | 4444 | 4444 | 4444 | 4444 | 3434 | 3423 | 3223 | 3322 | 2221 | 1122 | 1111 |

TABLE 5
Testing the virucidal properties of extracts against Adenovirus 36

| | | | Contact | | | | | | | | | | | | |
|---|---|---|---|---|---|---|---|---|---|---|---|---|---|---|---|
| | | | time | Dilution | | | | | | | | | | | |
| | | Conc. | (min) | 1 | 2 | 3 | 4 | 5 | 6 | 7 | 8 | 9 | 10 | 11 | 12 |
| Inoculated | Product | 0.1% | 60 | tttt | tttt | 4334 | 3422 | 3322 | 0000 | 0000 | 0000 | 0000 | 0000 | 0000 | 0000 |
| zabrus; | | | | tttt | tttt | 4433 | 4333 | 2211 | 0000 | 0000 | 0000 | 0000 | 0000 | 0000 | 0000 |
| isotonic | Virus | n.d | 0 | 4444 | 4444 | 4444 | 4444 | 4444 | 4444 | 4334 | 3334 | 3332 | 3222 | 2211 | 1101 |
| | control | | | 4444 | 4444 | 4444 | 4444 | 4444 | 4444 | 4333 | 3342 | 3322 | 2221 | 2111 | 0000 |
| | | n.d | 60 | 4444 | 4444 | 4444 | 4444 | 4444 | 4444 | 4433 | 3433 | 3332 | 2212 | 1121 | 1000 |
| | | | | 4444 | 4444 | 4444 | 4444 | 4444 | 4444 | 3344 | 2333 | 2233 | 2231 | 1121 | 0010 |

TABLE 6
Testing the virucidal properties of extracts against herpes simplex virus 1, and isotonic plasma control

| | | | Contact | | | | | | | | | | | | |
|---|---|---|---|---|---|---|---|---|---|---|---|---|---|---|---|
| | | | time | Dilution | | | | | | | | | | | |
| | | Conc. | (min) | 1 | 2 | 3 | 4 | 5 | 6 | 7 | 8 | 9 | 10 | 11 | 12 |
| Inoculated | Product | 0.1% | 60 | tttt | tttt | 4344 | 4343 | 4332 | 3222 | 2111 | 0000 | 0000 | 0000 | 0000 | 0000 |
| zabrus; | | | | tttt | tttt | 4434 | 4433 | 4333 | 3333 | 2111 | 0000 | 0000 | 0000 | 0000 | 0000 |
| isotonic | Virus | n.d | 0 | 4444 | 4444 | 4444 | 4444 | 4444 | 3444 | 3343 | 3233 | 3232 | 2232 | 2122 | 1111 |
| | control | | | 4444 | 4444 | 4444 | 4444 | 4444 | 3433 | 3323 | 3223 | 3222 | 3222 | 1222 | 2112 |
| | | n.d | 60 | 4444 | 4444 | 4444 | 4444 | 4444 | 3444 | 3344 | 3333 | 2233 | 2223 | 2212 | 2221 |
| | | | | 4444 | 4444 | 4444 | 4444 | 4444 | 4434 | 3423 | 3223 | 3323 | 2223 | 2222 | 1121 |
| Isotonic | Product | 0.1% | 60 | 4444 | 3444 | 3312 | 0001 | 0000 | 0000 | 0000 | 0000 | 0000 | 0000 | 0000 | 0000 |
| marine | | | | 4444 | 3344 | 2321 | 1000 | 0000 | 0000 | 0000 | 0000 | 0000 | 0000 | 0000 | 0000 |
| plasma | Virus | n.d | 0 | 4444 | 4444 | 4444 | 4444 | 4444 | 3323 | 0000 | 0000 | 0000 | 0000 | 0000 | 0000 |
| only | control | | | 4444 | 4444 | 4444 | 4444 | 4344 | 2223 | 0000 | 0000 | 0000 | 0000 | 0000 | 0000 |
| | | n.d | 60 | 4444 | 4444 | 4444 | 4444 | 3444 | 3222 | 0000 | 0000 | 0000 | 0000 | 0000 | 0000 |
| | | | | 4444 | 4444 | 4444 | 4444 | 4444 | 3222 | 0000 | 0000 | 0000 | 0000 | 0000 | 0000 |

TABLE 7
Testing the virucidal properties of extracts against
influenza virus A/H1N1 in embryonated chicken eggs

| | | Contact | | | | | | | | | | |
|---|---|---|---|---|---|---|---|---|---|---|---|---|
| | | time | Dilutions | | | | | | | | | |
| | Conc. | (min) | 1:2 | 1:4 | 1:8 | 1:16 | 1:32 | 1:64 | 1:128 | 1:256 | 1:516 | 1:1032 |
| Allantoic fluid from | 0.1% | 60 | − | − | − | − | − | − | − | − | − | − |
| ECEs inoculated with | | 60 | − | − | − | − | − | − | − | − | − | − |
| inoculated zabrus | | 60 | − | − | − | − | − | − | − | − | − | − |
| composition, isotonic, | | | | | | | | | | | | |
| and virus | | | | | | | | | | | | |
| Allantoic fluid from | n.d. | n.d. | + | + | + | + | + | + | − | − | − | − |
| ECEs inoculated with | | | + | + | + | + | + | + | − | − | − | − |
| virus | | | + | + | + | + | + | + | − | − | − | − |

TABLE 8
Testing the virucidal properties of extracts against human coronavirus OC43

| | | | Contact | | | | | | | | | | | | |
|---|---|---|---|---|---|---|---|---|---|---|---|---|---|---|---|
| | | | time | Dilution | | | | | | | | | | | |
| | | Conc. | (min) | 1 | 2 | 3 | 4 | 5 | 6 | 7 | 8 | 9 | 10 | 11 | 12 |
| Inoculated | Product | 3% | 60 | tttt | tttt | 0000 | 0000 | 0000 | 0000 | 0000 | 0000 | 0000 | 0000 | 0000 | 0000 |
| zabrus; | | | | tttt/ | tttt/ | 0000 | 0000 | 0000 | 0000 | 0000 | 0000 | 0000 | 0000 | 0000 | 0000 |
| isotonic | | | | 2222 | 1221 | | | | | | | | | | |
| | | | | 3222 | 1121 | | | | | | | | | | |
| | Virus | n.d | 0 | 4444 | 4334 | 3332 | 3322 | 2211 | 1111 | 0000 | 0000 | 0000 | 0000 | 0000 | 0000 |
| | control | | | 4444 | 4333 | 4432 | 3211 | 2221 | 1111 | 0000 | 0000 | 0000 | 0000 | 0000 | 0000 |
| | | n.d | 60 | 4444 | 4333 | 3324 | 3342 | 2222 | 1111 | 0000 | 0000 | 0000 | 0000 | 0000 | 0000 |
| | | | | 4444 | 4434 | 3333 | 2233 | 1212 | 1111 | 0000 | 0000 | 0000 | 0000 | 0000 | 0000 |

TABLE 9
Testing the virucidal properties of extracts extracts against Adenovirus 36

| | | | Contact | | | | | | | | | | | | |
|---|---|---|---|---|---|---|---|---|---|---|---|---|---|---|---|
| | | | time | Dilution | | | | | | | | | | | |
| | | Conc. | (min) | 1 | 2 | 3 | 4 | 5 | 6 | 7 | 8 | 9 | 10 | 11 | 12 |
| Inoculated | Product | 10% | 60 | tttt | tttt | tttt | 3432 | 2222 | 2211 | 0000 | 0000 | 0000 | 0000 | 0000 | 0000 |
| zabrus; | | | | tttt | tttt | tttt | 4322 | 2222 | 1101 | 0000 | 0000 | 0000 | 0000 | 0000 | 0000 |
| isotonic | Virus | n.d | 0 | 4444 | 4444 | 4444 | 4444 | 4444 | 4444 | 4433 | 3333 | 2232 | 2121 | 0000 | 0000 |
| | control | | | 4444 | 4444 | 4444 | 4444 | 4444 | 4344 | 4343 | 3332 | 3232 | 1111 | 0000 | 0000 |
| | | n.d | 60 | 4444 | 4444 | 4444 | 4444 | 4444 | 4444 | 4333 | 3333 | 3222 | 1112 | 0000 | 0000 |
| | | | | 4444 | 4444 | 4444 | 4444 | 4444 | 444 | 3334 | 2233 | 3233 | 2111 | 0000 | 0000 |

TABLE 10
Testing the virucidal properties of extracts against herpes simplex virus 1

| | | | Contact | | | | | | | | | | | | |
|---|---|---|---|---|---|---|---|---|---|---|---|---|---|---|---|
| | | | time | Dilution | | | | | | | | | | | |
| | | Conc. | (min) | 1 | 2 | 3 | 4 | 5 | 6 | 7 | 8 | 9 | 10 | 11 | 12 |
| Non- | Product | 10% | 60 | 4444 | 4444 | 4444 | 4444 | 4443 | 4432 | 3221 | 0000 | 0000 | 0000 | 0000 | 0000 |
| inoculated | C₀ | | | | | | | | | | | | | | |
| zabrus; | Product | 1% | 60 | 4444 | 4444 | 4444 | 4444 | 4434 | 4344 | 3222 | 2211 | 0000 | 0000 | 0000 | 0000 |
| isotonic | C₁ | | | | | | | | | | | | | | |
| | Product | 0.1% | 60 | 4444 | 4444 | 4444 | 4444 | 4444 | 4433 | 3333 | 3323 | 2222 | 2121 | 0000 | 0000 |
| | C₂ | | | | | | | | | | | | | | |
| | Product | 0.01% | 60 | 4444 | 4444 | 4444 | 4444 | 3444 | 4344 | 3332 | 3322 | 3222 | 2212 | 0000 | 0000 |
| | C₃ | | | | | | | | | | | | | | |
| | Product | 0.001% | 60 | 4444 | 4444 | 4444 | 4344 | 4444 | 4433 | 3433 | 2333 | 2322 | 2111 | 0000 | 0000 |
| | C₄ | | | | | | | | | | | | | | |
| | Virus | n.d | 60 | 4444 | 4444 | 4444 | 4444 | 4444 | 4443 | 3443 | 3332 | 3232 | 2212 | 0000 | 0000 |
| | control | | | | | | | | | | | | | | |

TABLE 11
Testing the virucidal properties of filtered extracts against herpes simplex virus 1

| | | | Contact | | | | | | | | | | | | |
|---|---|---|---|---|---|---|---|---|---|---|---|---|---|---|---|
| | | | time | Dilution | | | | | | | | | | | |
| | | Conc. | (min) | 1 | 2 | 3 | 4 | 5 | 6 | 7 | 8 | 9 | 10 | 11 | 12 |
| Non- | Product | 10% | 60 | 4444 | 4444 | 4444 | 4444 | 4343 | 3422 | 2121 | 1121 | 0000 | 0000 | 0000 | 0000 |
| inoculated | C₀ | | | | | | | | | | | | | | |
| zabrus; | Product | 1% | 60 | 4444 | 4444 | 4444 | 4344 | 3434 | 3344 | 3211 | 1211 | 0000 | 0000 | 0000 | 0000 |
| isotonic | C₁ | | | | | | | | | | | | | | |
| | Virus | n.d | 60 | 4444 | 4444 | 4444 | 4444 | 4444 | 3444 | 3443 | 3433 | 3232 | 3212 | 2211 | 1111 |
| | control | | | | | | | | | | | | | | |

1. A composition comprising a bee-derived component and a marine plasma, wherein the marine plasma comprises or consists of seawater.

2. The composition of claim 1, wherein the bee-derived component comprises or is derived from one or more of zabrus, honeycomb, bee venom, honey, royal jelly, propolis and pollen.

3. (canceled)

4. The composition of claim 2, wherein the bee-derived component comprises or is derived from zabrus.

5. (canceled)

6. The composition of claim 2, wherein the bee-derived component has a reduced content of hydrophobic components and/or sugars compared to the zabrus, honeycomb, bee venom, honey, royal jelly, propolis and/or pollen.

7. (canceled)

8. The composition of claim 2, wherein the bee-derived component comprises lysozyme.

9. The composition of claim 1, wherein the marine plasma comprises seawater diluted with water.

10. The composition of claim 9, wherein the seawater and water are present in relative amounts of approximately 25% to 30% seawater, and approximately 70% to 75% water.

11. The composition of claim 1, wherein the composition is formulated as a nasal spray, injectable serum, dermal patch, eyedrops and/or for oral administration.

12. A method of making a composition, the method comprising providing a bee-derived component and combining the bee-derived component with a marine plasma, wherein the marine plasma comprises or consists of seawater.

13. The method of claim 12, wherein the method comprises diluting the marine plasma with water.

14. (canceled)

15. The method of claim 12, wherein the method comprises obtaining the seawater from below one or more plankton blooms in the sea.

16. The method of claim 15, wherein the method further comprises filtering.

17. (canceled)

18. The method of claim 12, wherein the bee-derived component comprises or is derived from one or more of zabrus, honeycomb, bee venom, honey, royal jelly, propolis and pollen.

19. The method of claim 12, wherein the bee-derived component comprises or is derived from zabrus.

20. The method of claim 18, wherein the method comprises processing the one or more of zabrus, honeycomb, bee venom, honey, royal jelly, propolis and pollen to remove at least a portion of the hydrophobic components thereof and/or sugar content thereof.

21.-23. (canceled)

24. The method of claim 12, wherein the method comprises rearing bees as a source of bee-derived components.

25. The method of claim 24, wherein the method comprises providing the bees with a feed comprising farnesol and/or linden nectar.

26. (canceled)

27. The method of claim 24, wherein the method comprises rearing the bees in a beehive and treating the beehive with a fortification method.

28. The method of claim 27, wherein the fortification method comprises the application of rosin or a coating comprising rosin on the outside of the beehive.

29. The method of claim 24, wherein the method comprises obtaining one or more of zabrus, honeycomb, bee venom, honey, royal jelly, propolis and pollen from the bees which have been reared.

30. (canceled)

31. The method of claim 24, wherein the method comprises inoculating the bees against one or more specific pathogens before obtaining the bee-derived component.

32.-37. (canceled)

38. A method for treatment or protection against bacterial, viral, fungal, and/or parasitic diseases, the method comprising administering to a subject in need thereof an effective amount of a composition according to claim 1, or a composition produced according to the method of claim 12.

39.-46. (canceled)

47. The composition of claim 1, wherein the marine plasma has a mineral content according to:

| | |
|---|---|
| Aluminium Al 20.33 ppb | Manganese Mn 33.66 ppb |
| Antimony Sb 0.05 ppb | Mercury Hg ND |
| Arsenic As 8.23 ppb | Molybdenum Mo 5.33 ppb |
| Sulfur S 310.66 ppm | Neodymium Nd ND |
| Barium Ba 1.46 ppb | Niobium Nb 00233 ppb |
| Beryllium Be ND | Nickel Ni 17.76 ppb |
| Bismuth Bi ND | Gold Au ND |
| Boron B 2 ppm | Osmium Os 0.053 ppb |
| Bromine Br 23.66 ppm | Palladium Pd 4.33 ppb |
| Cadmium Cd 0.106 ppb | Silver Ag 0.436 ppb |
| Calcium Ca 134 ppm | Platinum Pt ND |
| Cerium Ce ND | Lead Pb 0.2566 ppb |
| Cesium Cs 0.109 ppb | Potassium K 130.66 ppm |
| Zirconium Zr 0.146 ppb | Praseodymium Pr ND |
| Cobalt Co 0.466 ppb | Rhenium Re ND |
| Copper Cu 1.03 ppb | Rhodium Rh 0.266 ppb |
| Chromium Cr 1.033 ppb | Rubidium Rb 38.33 ppb |
| Dysprosium Dy 0.0033 ppb | Ruthenium Ru ND |
| Erbium Er ND | Samarium Sm ND |
| Scandium Sc ND | Selenium Se 0.533 ppb |
| Tin Sn 0.6 ppb | Silicon Si 3.333 ppm |
| Strontium Sr 0.133 ppm | Sodium Na 1347.33 ppm |
| Europium Eu ND | Thallium Tl 0.00566 ppb |
| Phosphorus P 1.633 ppb | Tantalum Ta 0.0133 ppb |
| Gadolinium Gd ND | Tellurium Te ND |
| Gallium Ga 0.133 ppb | Terbium Tb ND |
| Germanium Ge 2.53 ppb | Titanium Ti 5.666 ppb |
| Hafnium Hf 0.023 ppb | Thorium Th ND |
| Iron Fe 12.26 ppb | Thulium Tm ND |
| Holmium Ho ND | Uranium U 1 ppb |
| Indium In ND | Vanadium V 9.66 ppb |
| Iridium Ir ND | Tungsten W 0.12 ppb |
| Lanthanum La 0.0076 ppb | Iodine I 7.66 ppb |
| Lithium Li 62 ppb | Ytterbium Yb ND |
| Lutetium Lu ND | Yttrium Y 0.033 ppb |
| Magnesium Mg 442.33 ppm | Zinc Zn 7.233 ppb |

or

| | |
|---|---|
| Aluminium Al 61 ppb | Manganese Mn 101 ppb |
| Antimony Sb 0.15 ppb | Mercury Hg ND |
| Arsenic As 24.7 ppb | Molybdenum Mo 16 ppb |
| Sulfur S 932 ppm | Neodymium Nd ND |
| Barium Ba 4.4 ppb | Niobium Nb 0.07 ppb |
| Beryllium Be ND | Nickel Ni 53.3 ppb |
| Bismuth Bi ND | Gold Au ND |
| Boron B 6 ppm | Osmium Os 0.16 ppb |
| Bromine Br 71 ppm | Palladium Pd 13 ppb |
| Cadmium Cd 0.32 ppb | Silver Ag* 1.31 ppb |
| Calcium Ca 402 ppm | Platinum Pt* ND |
| Cerium Ce ND | Lead Pb 0.77 ppb |
| Cesium Cs 0.327 ppb | Potassium K 392 ppm |
| Zirconium Zr 0.44 ppb | Praseodymium Pr ND |
| Cobalt Co 1.4 ppb | Rhenium Re ND |
| Copper Cu 13.8 ppb | Rhodium Rh 0.80 ppb |
| Chromium Cr 3.1 ppb | Rubidium Rb 115 ppb |
| Dysprosium Dy 0.010 ppb | Ruthenium Ru ND |
| Erbium Er ND | Samarium Sm ND |
| Scandium Sc ND | Selenium Se 1.6 ppb |
| Tin Sn 1.8 ppb | Silicon Si 10 ppm |
| Strontium Sr 9.4 ppm | Sodium Na 4042 ppm |
| Europium Eu ND | Thallium Tl 0.017 ppb |
| Phosphorus P 4.9 ppb | Tantalum Ta 0.04 ppb |
| Gadolinium Gd ND | Tellurium Te ND |
| Gallium Ga 0.40 ppb | Terbium Tb ND |
| Germanium Ge 7.6 ppb | Titanium Ti* 17 ppb |
| Hafnium Hf 0.07 ppb | Thorium Th ND |
| Iron Fe 36.8 ppb | Thulium Tm ND |
| Holmium Ho ND | Uranium U 3 ppb |
| Indium In ND | Vanadium V 29 ppb |
| Iridium Ir ND | Tungsten W 0.36 ppb |
| Lanthanum La 0.023 ppb | Iodine U 23 ppb |
| Lithium Li 184 ppb | Ytterbium Yb ND |
| Lutetium Lu ND | Yttrium Y 0.10 ppb |
| Magnesium Mg 1327 ppm | Zinc Zn 21.7 ppb |

48. The method of claim 12, wherein the marine plasma has a mineral content according to:

| | |
|---|---|
| Aluminium Al 20.33 ppb | Manganese Mn 33.66 ppb |
| Antimony Sb 0.05 ppb | Mercury Hg ND |
| Arsenic As 8.23 ppb | Molybdenum Mo 5.33 ppb |
| Sulfur S 310.66 ppm | Neodymium Nd ND |
| Barium Ba 1.46 ppb | Niobium Nb 00233 ppb |
| Beryllium Be ND | Nickel Ni 17.76 ppb |
| Bismuth Bi ND | Gold Au ND |
| Boron B 2 ppm | Osmium Os 0.053 ppb |
| Bromine Br 23.66 ppm | Palladium Pd 4.33 ppb |
| Cadmium Cd 0.106 ppb | Silver Ag 0.436 ppb |
| Calcium Ca 134 ppm | Platinum Pt ND |
| Cerium Ce ND | Lead Pb 0.2566 ppb |
| Cesium Cs 0.109 ppb | Potassium K 130.66 ppm |
| Zirconium Zr 0.146 ppb | Praseodymium Pr ND |
| Cobalt Co 0.466 ppb | Rhenium Re ND |
| Copper Cu 1.03 ppb | Rhodium Rh 0.266 ppb |
| Chromium Cr 1.033 ppb | Rubidium Rb 38.33 ppb |
| Dysprosium Dy 0.0033 ppb | Ruthenium Ru ND |
| Erbium Er ND | Samarium Sm ND |
| Scandium Sc ND | Selenium Se 0.533 ppb |
| Tin Sn 0.6 ppb | Silicon Si 3.333 ppm |
| Strontium Sr 0.133 ppm | Sodium Na 1347.33 ppm |
| Europium Eu ND | Thallium Tl 0.00566 ppb |
| Phosphorus P 1.633 ppb | Tantalum Ta 0.0133 ppb |
| Gadolinium Gd ND | Tellurium Te ND |
| Gallium Ga 0.133 ppb | Terbium Tb ND |
| Germanium Ge 2.53 ppb | Titanium Ti 5.666 ppb |
| Hafnium Hf 0.023 ppb | Thorium Th ND |
| Iron Fe 12.26 ppb | Thulium Tm ND |
| Holmium Ho ND | Uranium U 1 ppb |
| Indium In ND | Vanadium V 9.66 ppb |
| Iridium Ir ND | Tungsten W 0.12 ppb |
| Lanthanum La 0.0076 ppb | Iodine I 7.66 ppb |
| Lithium Li 62 ppb | Ytterbium Yb ND |
| Lutetium Lu ND | Yttrium Y 0.033 ppb |
| Magnesium Mg 442.33 ppm | Zinc Zn 7.233 ppb |

or

| | |
|---|---|
| Aluminium Al 61 ppb | Manganese Mn 101 ppb |
| Antimony Sb 0.15 ppb | Mercury Hg ND |
| Arsenic As 24.7 ppb | Molybdenum Mo 16 ppb |
| Sulfur S 932 ppm | Neodymium Nd ND |
| Barium Ba 4.4 ppb | Niobium Nb 0.07 ppb |
| Beryllium Be ND | Nickel Ni 53.3 ppb |
| Bismuth Bi ND | Gold Au ND |
| Boron B 6 ppm | Osmium Os 0.16 ppb |
| Bromine Br 71 ppm | Palladium Pd 13 ppb |
| Cadmium Cd 0.32 ppb | Silver Ag* 1.31 ppb |
| Calcium Ca 402 ppm | Platinum Pt* ND |
| Cerium Ce ND | Lead Pb 0.77 ppb |
| Cesium Cs 0.327 ppb | Potassium K 392 ppm |
| Zirconium Zr 0.44 ppb | Praseodymium Pr ND |
| Cobalt Co 1.4 ppb | Rhenium Re ND |
| Copper Cu 13.8 ppb | Rhodium Rh 0.80 ppb |
| Chromium Cr 3.1 ppb | Rubidium Rb 115 ppb |
| Dysprosium Dy 0.010 ppb | Ruthenium Ru ND |
| Erbium Er ND | Samarium Sm ND |
| Scandium Sc ND | Selenium Se 1.6 ppb |
| Tin Sn 1.8 ppb | Silicon Si 10 ppm |
| Strontium Sr 9.4 ppm | Sodium Na 4042 ppm |
| Europium Eu ND | Thallium Tl 0.017 ppb |
| Phosphorus P 4.9 ppb | Tantalum Ta 0.04 ppb |
| Gadolinium Gd ND | Tellurium Te ND |
| Gallium Ga 0.40 ppb | Terbium Tb ND |
| Germanium Ge 7.6 ppb | Titanium Ti* 17 ppb |
| Hafnium Hf 0.07 ppb | Thorium Th ND |
| Iron Fe 36.8 ppb | Thulium Tm ND |
| Holmium Ho ND | Uranium U 3 ppb |
| Indium In ND | Vanadium V 29 ppb |
| Iridium Ir ND | Tungsten W 0.36 ppb |
| Lanthanum La 0,023 ppb | Iodine I 23 ppb |
| Lithium Li 184 ppb | Ytterbium Yb ND |
| Lutetium Lu ND | Yttrium Y 0.10 ppb |
| Magnesium Mg 1327 ppm | Zinc Zn 21.7 ppb. |

49. The method of claim 38, wherein the viral disease is mediated by a poliovirus, adenovirus, coronavirus or herpesvirus.

50. The method of claim 38, wherein the method comprises intranasal, intramuscular, intravenous, intraarterial, subcutaneous, intraperitoneal, topical, transmucosal, and/or oral administration.

51. The method of claim 38, wherein the composition is a vaccine.

52. The composition of claim 10, wherein the seawater and water are present in relative amounts of approximately 29% seawater, and approximately 71% water.

53. The method of claim 12, wherein the method comprises obtaining the seawater from between about 25 and about 35 metres below one or more plankton blooms in the sea.

54. The method of claim 12, wherein the method comprises obtaining the seawater from around 30 metres below one or more plankton blooms in the sea.
